(19) United States
(12) Patent Application Publication
(10) Pub. No.: US 2014/0170724 A1
(43) Pub. Date: Jun. 19, 2014

(54) REPLICATING EXPRESSION VECTOR AND METHODS

(71) Applicants: JANET WESTPHELING, BOGART, GA (US); DAEHWAN CHUNG, ATHENS, GA (US)

(72) Inventors: JANET WESTPHELING, BOGART, GA (US); DAEHWAN CHUNG, ATHENS, GA (US)

(73) Assignee: UNIVERSITY OF GEORGIA RESEARCH FOUNDATION, INC., ATHENS, GA (US)

(21) Appl. No.: 14/134,714

(22) Filed: Dec. 19, 2013

Related U.S. Application Data
(60) Provisional application No. 61/739,393, filed on Dec. 19, 2012.

(51) Int. Cl. C12N 15/74; C12P 7/54; C12P 7/56; C12P 7/10

(52) U.S. Cl. CPC C12N 15/74; C12P 7/10; C12P 7/54; C12P 7/56
USPC 435/165; 435/320.1; 435/471

(57) ABSTRACT

This disclosure provides a shuttle vector for transferring genetic material between Caldicellulosiruptor spp. and an amplification cell. Generally, the shuttle vector includes an origin of replication sequence from the amplification cell, an origin of replication for Caldicellulosiruptor spp., a selectable marker for the amplification cell, and a heterologous coding sequence that complements a functional deletion in the Caldicellulosiruptor spp. genome. Also disclosed are genetically modified cells that include such a vector, and methods of making and using such shuttle vectors and genetically modified cells.

### CROSS-REFERENCE TO RELATED APPLICATION

This application claims priority to U.S. Provisional Patent Application Ser. No. 61/739,393, filed Dec. 19, 2012, which is incorporated herein by reference.

### GOVERNMENT FUNDING

This invention was made with government support under DE-AC05-00OR22725 awarded by the BioEnergy Science Center of the Department of Energy and 5T32GM007103-30 awarded by the National Institutes of Health. The government has certain rights in the invention.

### SEQUENCE LISTING

This application contains a Sequence Listing electronically submitted via EFS-Web to the United States Patent and Trademark Office as an ASCII text filed entitled “235-02200101_SequenceListing_ST25.txt” having a size of 12 kilobytes and created on Dec. 9, 2013. The information contained in the Sequence Listing is incorporated by reference herein.

### SUMMARY

This disclosure provides, in one aspect, a shuttle vector for transferring genetic material between Caldicellulosiruptor spp. and an amplification cell. Generally, the shuttle vector includes an origin of replication sequence from the amplification cell, an origin of replication for Caldicellulosiruptor spp., a selectable marker for the amplification cell, and a heterologous coding sequence that complements a functional deletion in the Caldicellulosiruptor spp. genome.

In some embodiments, the amplification cell can be E. coli.

In some embodiments, the selectable marker can include antibiotic resistance.

In some embodiments, the Caldicellulosiruptor spp. can be C. bescii.

In some embodiments, the heterologous coding sequence can include pyrF operably linked to a regulatory sequence.

In some embodiments, the heterologous coding sequence can include a coding region from a species different than the Caldicellulosiruptor spp. In some of these embodiments, the heterologous coding sequence can encode a polypeptide involved in plant biomass deconstruction. In some embodiments, the polypeptide encoded by the heterologous coding sequence is involved in biosynthesis of a biofuel. In some embodiments, the polypeptide encoded by the heterologous coding sequence is involved in biosynthesis of a bioproduct.

In another aspect, this disclosure provides a method that generally includes introducing the shuttle vector as summarized immediately above into a cell. In another aspect, this disclosure provides a genetically modified cell that includes such a shuttle vector.

In another aspect, this disclosure describes a genetically modified Caldicellulosiruptor spp. microbe engineered to increase biosynthesis of a bioproduct, wherein the genetically modified Caldicellulosiruptor spp. microbe exhibits an increase in biosynthesis of the bioproduct compared to a wild type control. In some embodiments, the bioproduct can include a biofuel. In some of these embodiments, the biofuel can include ethanol.

In some embodiments, the genetically modified Caldicellulosiruptor spp. microbe exhibits an increase in biosynthesis of the bioproduct compared to a wild type control when grown on lignocellulosic biomass. In some of these embodiments, the lignocellulosic biomass can include switchgrass.

In yet another aspect, this disclosure provides a method that generally includes growing a genetically modified Caldicellulosiruptor spp. microbe on a feed stock effective for the genetically modified Caldicellulosiruptor spp. microbe to biosynthesize the bioproduct.

In some embodiments, the bioproduct can include a biofuel. In some of these embodiments, the biofuel can include ethanol.

In some embodiments, the feed stock can include lignocellulosic biomass. In some of these embodiments, the lignocellulosic biomass can include switchgrass.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Chromosomal map and PCR analysis of the Uridine Monophosphate (UMP) biosynthetic gene cluster in C. bescii DSM 6725 and the spontaneous deletion in pyrFA (JWCB005) locus. (A) A diagram of the pyr operon region with the 878 bp deletion in the pyrFA ORFs. The line below the diagram indicates the length of the deletion. Bent arrows depict primers used for verification of the structure of the chromosome in the JWCB005 (ΔpyrFA) strain. pyrF and pyrE loci indicated as black color filled arrow and black dashed filled arrow, respectively. (B) Gel depicting PCR products of the pyrFA region in wild type (3.44 kb) compared to the ΔpyrFA (2.52 kb) strain amplified by primers (JH020 and FJ298). (C) Gel depicting the 2.66 kb PCR products of pyrE region in wild type and the ΔpyrFA strain by primers (DC326 and DC331). M: 1 KB DNA ladder (New England Biolabs Inc., Ipswich, Mass.).

FIG. 2. Plasmid map of shuttle vector (pDCW89) and verification of its presence as a non-integrated, autonomous plasmid in C. bescii transformants. (A) A linear DNA fragment containing the pyrF expression cassette as well as the entire sequence of pBAS2, generated by PCR amplification using primers DC283 and DC284, was ligated to a DNA fragment containing E. coli replication and selection functions to generate the final shuttle vector. The cross-hatched box corresponds to the pBAS2 plasmid sequences. ORFs from C. bescii are indicated as empty arrows and those from E. coli as black arrows. The apramycin resistant cassette (Apr^{R}); PSC 101 low copy replication origin in E. coli; repA, a plasmid-encoded gene required for PSC101 replication; par, partition locus are indicated. The proposed replication origin (115 bp) of pBAS2 is indicated. The primers and restriction sites (AatII and EcoRI) used for the verification are indicated. A detailed description of the construction of pDCW89 is described in FIG. 5 and the Materials and Methods. (B) Gel showing the 1.6 kb PCR products containing the pSC101 ori sequences only presence in pDCW89 using primers DC230 and JF199, total DNA from JWCB005 (Lane 1), a C. bescii transformant with pDCW89 (Lane 2), and pDCW89 isolated from E. coli (Lane 3) as template. (C) Restriction analysis of plasmid DNA before and after transformation of C. bescii and back-transformation to E. coli. Lanes 1 and 4, pDCW89 plasmid DNA isolated from E. coli DH5α, and digested with AatII (Lane 1, 4.4 kb and 3.3 kb cleavage products), and EcoRI (Lane 4, 1.9 kb and 5.8 kb cleavage products); lane 2, 3, 5, 6, plasmid DNA isolated from two biologically independent E. coli DH5α back-transformed from C. bescii transformants, and digested with AatII (Lane 2 & 3), and EcoRI (Lane 5 & 6). M: 1 KB DNA ladder (New England Biolabs Inc., Ipswich, Mass.).

FIG. 3. Comparison of DNA modification status between shuttle vector DNA isolated from E. coli (Lane 1) and C. hydrothermalis transformants (Lane 2) by Restriction analysis. (A) Undigested, (B) Digested with HindIII (4.3 and 3.4 kb cleavage products); (C) Digested with EcoRI (4.6 and 1.9 kb cleavage products); (D) Digested with CbeI (11 cleavage products are expected). M: 1 KB DNA ladder.

FIG. 4. Plasmid map of pDCW129 and verification of its ability to stably maintain an inserted DNA fragment through transformation and replication in C. bescii. (A) Diagram of pDCW129. A linear DNA fragment containing the CBM3 and linker region derived from celA (Cbes1867) was inserted into pDCW89 shuttle vector. The cross-hatched box corresponds to a 0.68 kb of inserted DNA fragment. All features in pDCW129 are indicated at figure legend in FIG. 2A. The primers and restriction site (EcoRV) used for the construction and verification are indicated. (B) Gel showing the 2.2 kb DNA fragment containing the pyrF cassette and inserted DNA fragment, amplified by using primers DC233 and DC235. Lane 1, total DNA isolated from JWCB005; lane 2, total DNA isolated from C. bescii transformant with pDCW129; lane 3, pCW129 isolated from E. coli (C) EcoRV restriction digestion analysis of plasmid DNA before and after transformation of C. bescii and back-transformation to E. coli. Lane 1, pDCW129 plasmid DNA isolated from E. coli DH5α; lane 2, 3 and 4, plasmid DNA isolated from three biologically independent E. coli DH5α back-transformed from C. bescii transformants. M: 1 KB DNA ladder (New England Biolabs Inc., Ipswich, Mass.).

FIG. 5. Construction of shuttle vector pDCW89. The cross-hatched box corresponds to pBAS2 plasmid sequences. ORFs from C. bescii are indicated as empty arrows and those from E. coli as black arrows. The apramycin resistant cassette (Apr^{R}); PSC 101 low copy replication origin in E. coli; repA, a plasmid-encoded gene required for PSC 101 replication; par, partition locus; pyrF cassette are indicated. The proposed replication origin (115 bp) of pBAS2 is indicated. All primers and two restriction sites (KpnI and XhoI) used in this construction are also indicated.

FIG. 6. Determination of copy number and maintenance of pDCW89 in C. bescii. (A) Diagram of the pyrF chromosomal region. EcoRV sites (“E”) are indicated, as are the locations of primers used to generate the pyrF hybridization probe. (B) Southern blot of the pDCW89 transformant (JWCB011). Lanes 1 to 5, DNA isolated from 5 successive passages in non-selective medium; lanes 6 to 10, 5 successive passages in selective medium; lane 11, JWCB005; lane 12, C. bescii wild type; Lane 13, pDCW89 isolated from E. coli.

FIG. 7. Plasmid constructions to determine the minimal sequence requirement for replication in C. bescii. DNA sequences derived from C. bescii are indicated as empty arrows and boxes. The proposed replication origin (115 bp) of pBAS2 is indicated. All primers and two restriction sites (KpnI and PvuII) used in this construction are also indicated. (A) Diagram of pDCW154. (B) Diagram of pDCW155.

FIG. 8. (A) Depiction of “single step bioprocessing” and its advantages over conventional methods and the engineering of C. bescii for ethanol production directly from biomass. (B) (1) L-Lactate dehydrogenase; (2) Pyruvate-ferredoxin oxidoreductase; (3) Bifurcating (reduced ferredoxin:NADH-dependent) hydrogenase; (4) Acetaldehyde dehydrogenase; (5) Phosphotransacetylase; (6) Alcohol dehydrogenase; (7) Acetate kinase; (8) NADH-dependent aldehyde dehydrogenase.

FIG. 9. Targeted insertion and expression of C. thermocellum ATCC27405 adhE (Cthe0423) in C. bescii. (A) A diagram of the integrational vector pDCW144 (see FIG. 12 for details of plasmid construction), which contains the P_{S-layer} Cthe-adhE expression cassette and pyrF cassette (Chung et al., 2013 PLoS ONE 8:e62881) for selection of transformants. Homologous recombination can occur at the upstream or downstream targeted chromosomal regions, integrating the plasmid into the genome and generating a strain that is a uracil prototroph. Counter-selection with 5-fluoroorotic acid (5-FOA) selects for loss of the plasmid sequences but not the adhE expression cassette. Bent arrows depict primers used for verification of the integrated expression cassette (2.6 kb). Apr^{r}, apramycin resistance cassette. (B) Gel depicting PCR products amplified from the targeted chromosome region in JWCB018 (lane 1), JWCB032 (lane 2), and JWCB033 (lane 3) amplified by primers DC477 and DC478. M: 1 kb DNA ladder (New England Biolabs Inc., Ipswich, Mass.). (C) Western blot analysis of C. bescii strains used in this study. The 77 μg of total cell protein lysate isolated from the mid-log phase cultures grown at various temperatures either 60° C., 65° C., and 70° C. were electrophoresed and probed with His-tag antibody as described in Material and Methods Lane 1: JWCB018; lane 2: JWCB032; lane 3: JWCB033; M: MagicMark™ XP Western Protein Standard.

FIG. 10. Growth and analysis of fermentation products of C. bescii containing C. thermocellum adhE compared to wild type strains. Growth of C. bescii strains on 1.0% cellobiose as the carbon source at 65° C. (A) and 75° C. (B) (log₁₀ OD₆₈₀ₙₘ). Analysis of fermentation products lactate (C,F,I), acetate (D,G,J) and ethanol (E,H,K) after growth on cellobiose (1%, wt/v, C,D,E), avicel (2%, wt/v, F,G,H) and switchgrass (2%, wt/v, H,J,K) at 65° C. circles, JWCB001; squares, JWCB018; diamonds, JWCB032. Error bars based on two biologically independent experiments.

FIG. 11. Analysis of ethanol tolerance of the C. bescii wild-type DSM 6725. Growth of C. bescii on 1.0% cellobiose as the carbon source with different amounts of ethanol at (A) 65° C. and (B) 75° C. monitored by measuring culture turbidity (log₁₀ OD₆₈₀ₙₘ). Closed circles, no ethanol; open circles, 200 mM; closed squares, 300 mM; open squares, 400 mM; closed triangles, 450 mM; open triangles, 500 mM; closed diamonds, 600 mM; open diamonds, 700 mM. Error bars based on two biologically independent experiments.

FIG. 12. Construction of knock-in vector pDCW144. Plasmid pDCW144 was constructed in four cloning steps. ORFs from C. bescii and Clostridium thermocellum are indicated as empty arrows. ORFs from E. coli indicated as black arrows. The apramycin resistant cassette (Apr^{R}); pSC101, low copy replication origin in E. coli; repA, a plasmid-encoded gene required for pSC101 replication; par, partition locus; pyrF cassette; 5′ and 3′ franking sequences of the targeted insertion site in C. bescii chromosome; regulatory and rho independent terminator sequences surrounding Cbes2303 (marked as a cross-hatched box); C-terminal 6× Histidine-tag in front of stop codon are indicated. All primers and two restriction sites (BamHI and SphI) used in this construction are also indicated.

### DETAILED DESCRIPTION OF ILLUSTRATIVE EMBODIMENTS

The recalcitrance of plant biomass can be a barrier to economical microbial conversion of plant biomass to products of interest such as biofuels and other bioproducts. Thermophilic microbes including members of the genus Caldicellulosiruptor can grow on non-pretreated lignocellulosic biomass and can, therefore, be an attractive option for biomass conversion. The ability to genetically manipulate members of this genus can allow one to exploit the potential of these microbes for Consolidated BioProcessing (CBP) (Lynd et al., 2005 Curr Opin Biotechnol 16:577-583). The use of Caldicellulosiruptor species for CBP can be limited, however, by a lack of molecular biology tools that permit efficient transfer of genetic material between members of the genus Caldicellulosiruptor and other microbial species.

Replicating shuttle vectors facilitate a variety of genetic manipulations including, for example, optimizing transformation protocols and homologous and heterologous expression of genes of interest. For members of the Caldicellulosiruptor genus, a replicating shuttle vector can be an important tool for extending the breadth of biomass substrates on which the microbes can grow, the metabolic pathways that may be engineered into the microbes, and the products that may be produced by the engineered metabolic pathways. Most reported shuttle vectors rely on drug resistance markers for selection, but these markers are not suitable for use in hyperthermophiles like Caldicellulosiruptor species, which grow optimally at and above 70° C. Typically, the antibiotics and/or the corresponding resistance gene products are not stable at such temperatures.

This disclosure provides a replicating shuttle vector for Caldicellulosiruptor species based on pBAS2, the smaller of two native C. bescii plasmids. pBAS2 was constructed to contain the wild type pyrF allele for uracil prototrophic selection. This plasmid is capable of stable replication and selection in both E. coli and C. bescii. Plasmid DNA was unchanged during transformation and replication in C. bescii and back transformation into E. coli. Transformation with replicating plasmid DNA is an order of magnitude more efficient than transformation with non-replicating plasmids in C. bescii (Chung et al., 2012 PLoS One 7:e43844), making this vector an important tool to screen transformability of members of this genus.

A similar approach was used to screen for transformability of other members of this genus using M.CbeI to overcome restriction as a barrier and was successful for transforming C. hydrothermalis, an attractive species for many applications. Plasmids containing a carbohydrate binding domain (CBM) and linker region from the C. bescii celA coding region were maintained with selection and were structurally stable through transformation and replication in C. bescii and E. coli. Moreover, the translation product of celA is predicted to be heavily glycosylated. Since E. coli doesn't glycosylate proteins, the ability to examine CelA produced in E. coli as well as C. bescii is an example of the use of this shuttle vector for analysis of important biological functions unique to Caldicellulosiruptor spp.

Isolation of a Spontaneous Deletion of the C. bescii pyrFA Locus for Nutritional Selection of Transformants.

Attempts to use drug resistance markers for selection of transformants in C. bescii have been unsuccessful. We therefore turned our attention to an alternative selection marker. Orotidine monophosphate (OMP) decarboxylase, encoded by pyrF in bacteria (ura3 in yeast), converts the pyrimidine analog 5-fluoroorotic acid (5-FOA) to 5-fluorouridine monophosphate, which is ultimately converted to fluorodeoxyuridine by the uracil biosynthetic pathway. Fluorodeoxyuridine is a toxic product that kills growing cells that are synthesizing uracil (Bocke et al., 1984 Mol Gen Genet 197:345-346). Mutants of pyrF are, therefore, uracil auxotrophs and resistant to 5-FOA, providing uracil prototrophy as a selection for the wild type allele and 5-FOA resistance as a counter selection for the mutant allele. We previously reported the isolation and use of a pyrF mutant for nutritional selection of transformants, but the mutation involved a deletion of most of the pyrBCF region. Complementation of the mutation therefore required all three deleted coding regions. Cloning such a large region of the chromosome for complementation is undesirable for designing a replicating plasmid vector since increased size often results in lower transformation efficiency.

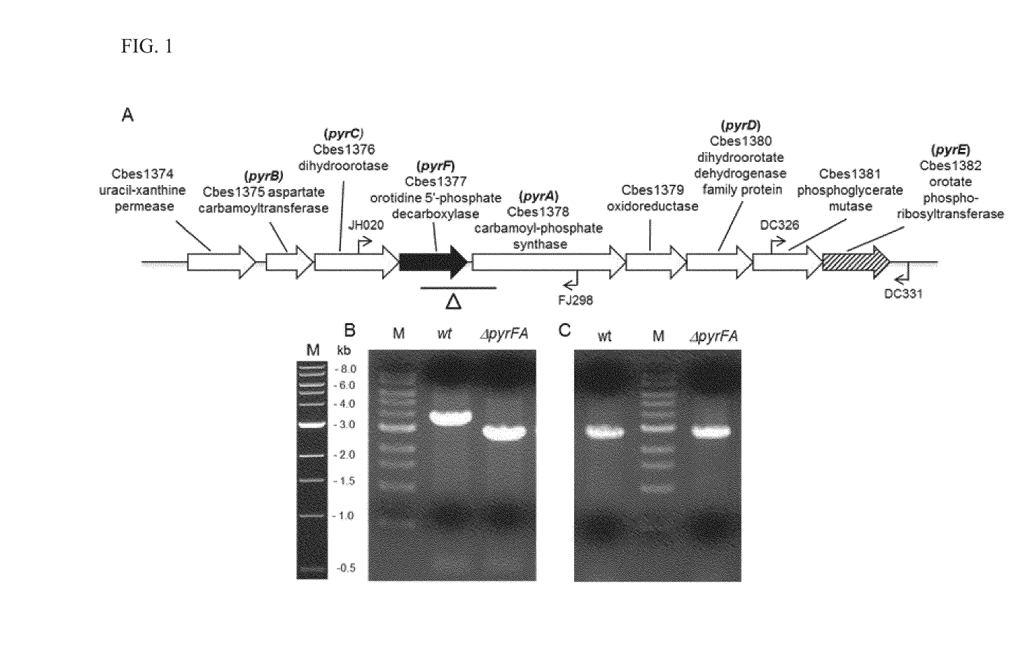

Here, we instead isolated a different deletion mutant, in the pyrFA locus, that was complemented by pyrF alone. The partial deletion of pyrA is unusual in that it can be complemented with only the wild-type pyrF allele. This simple complementation by a single coding region reduces the size of the elements required to successfully construct the shuttle vector since the entire operon is not needed to complement the mutation. To obtain this new deletion strain, C. bescii cells were plated on modified DSMZ 640 media (Chung et al., 2012 PLoS One 7:e43844) containing 8 mM 5-FOA. Spontaneous resistance to 5-FOA was observed at a frequency of approximately 10⁻⁵ at 65° C. Among 30 mutants isolated, one, designated JWCB005 (Table 1), had an 878 bp deletion that spans most of the pyrF open reading frame (Cbes1377), and part of the adjacent gene, pyrA (Cbes1378) (FIG. 1A). The extent of the deletion was defined by PCR amplification of the pyrFA region in the mutant and subsequent sequencing of the PCR product (FIG. 1B). We also PCR amplified and sequenced the pyrE region, also required for uracil biosynthesis, and found it to be wild type (FIG. 1C). JWCB005 was a tight uracil auxotroph capable of growth in media supplemented with uracil, but not orotate, confirming that pyrF function was absent in this deletion. The function of pyrA does not seem to be affected by the deletion because transformation with pDCW89, containing only the wild type pyrF allele, was able to complement the uracil auxotrophy without added orotate, which is the product of pyr4 in the uracil biosynthetic pathway. Reversion to uracil prototrophy was not a concern, making prototrophic selection possible no matter how low the frequency of transformation. Growth of the JWCB005 mutant supplemented with uracil (40 μM) was comparable to that of the wild type, reaching a cell density of approximately 2×10⁸ in 24 hours.

Construction of a Replicating Shuttle Vector Based on pBAS2.

C. bescii contains two native plasmids, pBAL (8.3 kb) and pBAS2 (3.7 kb) (Dam et al., 2011 Nucleic Acids Res 39:3240-3254; Clausen et al., 2004 Plasmid 52:131-138). Because of its relatively small size, we chose to use pBAS2 to supply replication functions for C. bescii in the shuttle vector. The pBAS2 plasmid was first reported nearly a decade ago, but the copy number was never determined. The plasmid contains sequences that show homology to a double stranded replication origin, characteristic of plasmids with rolling circle replication, however no single stranded intermediates of plasmid replication were detected (Clausen et al., 2004 Plasmid 52:131-138).

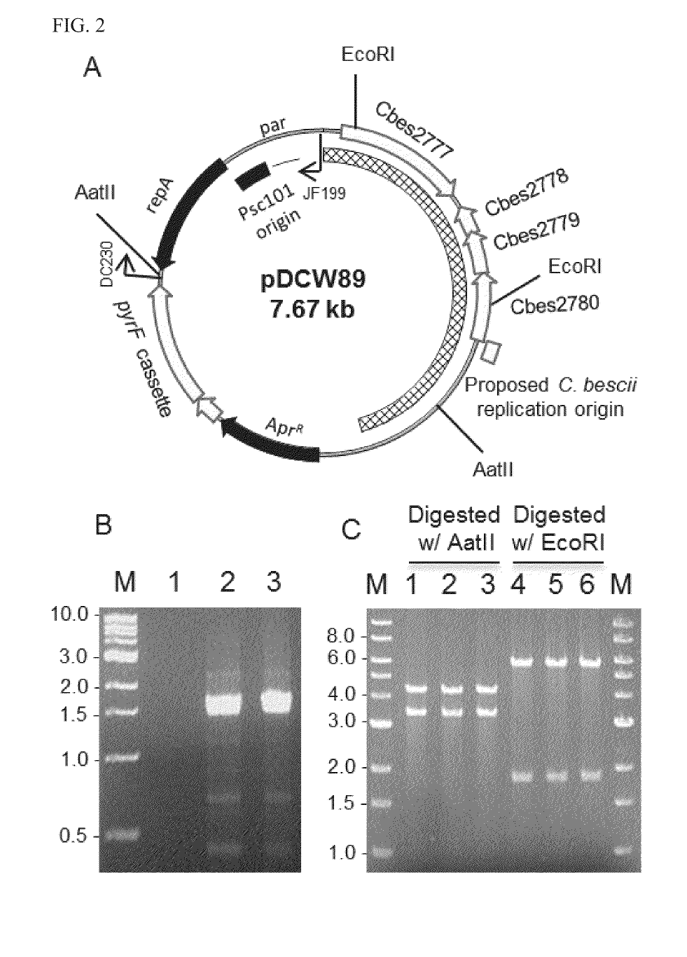

To avoid disrupting the replication functions of the pBAS2 plasmid, we linearized the plasmid DNA just upstream of the Cbes2777 ORF and inserted aac, a selection marker conferring apramycin resistance in E. coli. C. bescii pyrF, placed under transcriptional control of the promoter of Cbes2105 (30S ribosomal protein S30EA), was used for selection of uracil prototrophy in the C. bescii pyrFA deletion mutant JWCB005. The pSC101 replication origin was used for replication in E. coli. The resulting plasmid, pDCW89 (FIG. 2A), was transformed into C. bescii by electroporation, and cells were plated onto defined medium without uracil as described (Chung et al., 2012 PLoS One 7:e43844). Uracil prototrophic colonies were selected and transformation was confirmed by PCR amplification of a portion of the pSC101 replication origin present only in pDCW89 (FIG. 2B). Total DNA isolated from two biologically independent transformants was used to back-transform E. coli. Restriction digestion analysis showed that the plasmid was unchanged during transformation and replication in C. bescii and/or subsequent back transformation to E. coli (FIG. 2C). This result suggests that it was replicating autonomously in both organisms as an integrated vector would have produced a different restriction profile. The resulting strain was designated JWCB011 (Table 1). The transformation frequency varied between experiments, but was typically about 500 transformants per μg of plasmid DNA. This efficiency was 10 times higher than the transformation efficiency observed with non-replicating plasmids in C. bescii (Chung et al., 2012 PLoS One 7:e43844).

Assessment of Plasmid Maintenance, and Relative Copy Number in C. bescii.

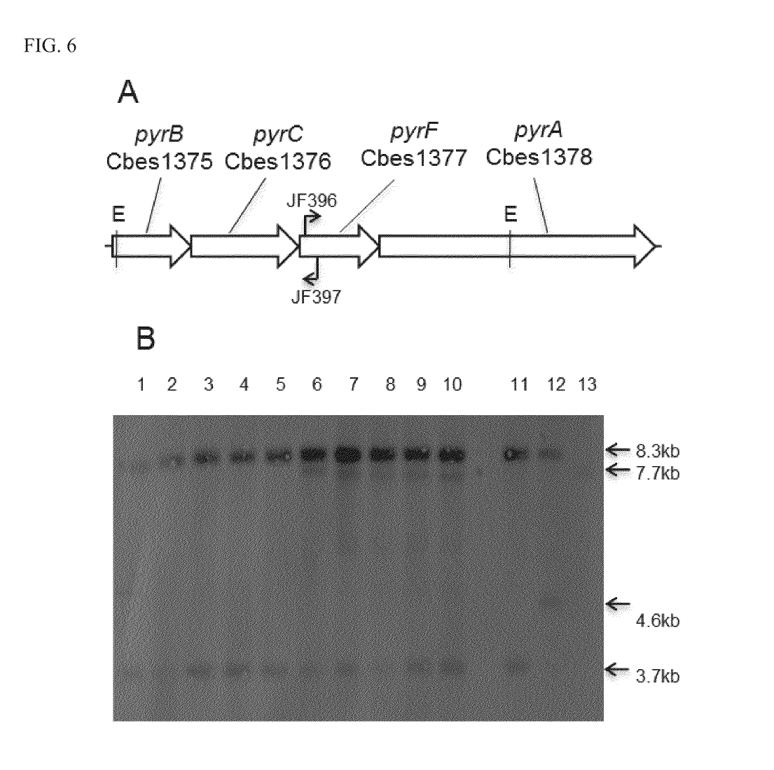

To assess plasmid maintenance and relative copy number, C. bescii transformants were serially sub-cultured every 16 hours for five passages in selective and nonselective liquid LOD medium (Farkas et al., 2012 J Ind Microbiol Biotechnol 40:41-49). Total DNA isolated from cells after each passage was used for Southern hybridization analysis (FIG. 6). To generate a probe for the detection of a sequence contained once on both the plasmid and the chromosome, primers JF396 and JF397 (Table 2) were used to amplify a fragment of pyrF remaining in the genome of JWCB005, and also contained on the plasmid. Relative copy number was determined as the ratio of band intensity of the plasmid-derived copy of the pyrF locus (7.7 kb) compared to the chromosome-derived copy of the pyrF locus (3.7 kb) in JWCB005 (FIG. 6). The relative intensity was 0.8 to 1.1 suggesting that the shuttle vector exists as a single copy per chromosome (FIG. 6).

Most plasmids that replicate via a rolling circle mechanism exist in high copy per chromosome (Espinosa et al., 1995 FEMS Microbiol Lett 130:111-120) and the native pBAS2 may exist in high copy as well. The relative copy number of pBAS2 was determined by qPCR with primer pairs targeting specific regions of pBAS2 and/or the chromosome. The relative copy number of pBAS2 was calculated to be seventy-five copies per chromosome based on two biologically independent analyses. The fact that the shuttle vector exists in a single copy per chromosome may be due, at least in part, to competition with the endogenous pBAS2 since they share replication and maintenance functions. The 4.6 kb band indicates the pyrF-containing fragment in wild type C. bescii (lane 12) and 8.3 kb band is non-specific hybridization with pBAL, the larger of two endogenous plasmids in C. bescii (FIG. 6).

Plasmid maintenance was determined by assessing the presence of the plasmid after passage with and without nutritional selection over the five successive transfers. Southern analysis showed that the plasmid relative copy number remains constant with selection, but that the plasmid is quickly lost without selection (FIG. 6, lanes 1-5). A single passage in nonselective media (with 40 M uracil) is enough for the plasmid to be lost from the majority of cells (FIG. 6, lane 1). Both the very low copy number and rapid loss without selection may be due to plasmid incompatibility between pBAS2 and pDCW89. This feature of the plasmid could be advantageous for genetic engineering applications that involve plasmid curing, eliminating the need for counter-selection with, for example, 5-FOA and/or another antimetabolite that may be potentially mutagenic. In certain applications, a single copy plasmid can provide certain advantages over high copy plasmids such as, for example, providing expression of genes at physiologically relevant levels.

Transformation of C. hydrothermalis with Shuttle Vector DNA Methylated with M.CbeI.

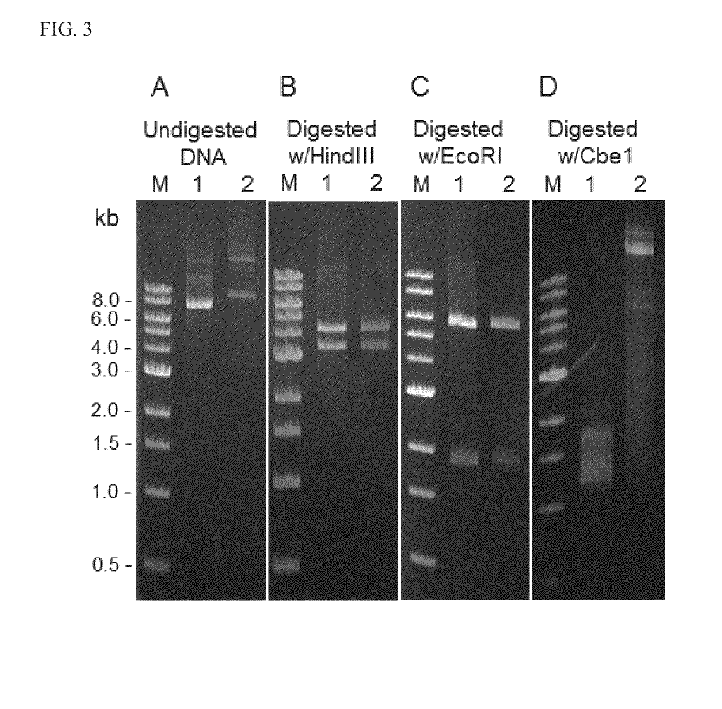

Restriction of transforming DNA is a barrier—and for C. bescii apparently an absolute barrier—to transformation of DNA from E. coli (Chung et al., 2012 PLoS One 7:e43844; Chung et al., 2011 J Ind Microbiol Biotechnol 38:1867-1877). Transformation of plasmid DNA from E. coli into C. bescii can involve in vitro methylation with an endogenous α-class N4-Cytosine methyltransferase. M.CbeI (Chung et al., 2012 PLoS One 7:e43844). To test whether modification by M.CbeI also allowed transformation of other members of this genus, a spontaneous mutation resistant to 5-FOA was isolated in C. hydrothermalis (Chung et al., 2013 J Ind Microbiol Biotechnol 40:517-521), JWCH003 (Table 1). This mutant was a tight uracil auxotroph and was used as a host for plasmid transformation. Unmethylated plasmid DNA isolated from various E. coli hosts failed to transform this mutant but DNA methylated with M.CbeI transformed at a frequency similar to that for C. bescii (typically about 500 transformants per μg of plasmid DNA). Transformants were initially confirmed by PCR amplification of aac contained exclusively on the plasmid. As shown in FIG. 3, restriction digestion analysis using HindIII and EcoRI of shuttle vector plasmid DNA isolated from C. hydrothermalis transformants was indistinguishable from that isolated from E. coli (FIGS. 3B and 3C), suggesting that it is structurally stable in C. hydrothermalis. This further suggests that modification with M.CbeI may have utility in DNA transformation of a variety of Caldicellulosiruptor species. Moreover, methylation by HindIII methyltransferase partially protects the plasmid in vitro but does not allow transformation. Only methylation with M.CbeI allows transformation. These data also provide evidence that the use of the wild type C. bescii pyrF allele under the control of the ribosomal protein S30EA promoter functions in at least one other species and will likely prove to be a useful selection marker for many species. This shuttle vector may, therefore, facilitate extension of genetic methods to a number of other Caldicellulosiruptor species.

Shuttle vector plasmid DNA was readily isolated from C. hydrothermalis, suggesting that the vector may exist in higher copy in C. hydrothermalis than in C. bescii. This may be due, at least in part, to the absence of a competing plasmid in C. hydrothermalis. FIG. 3A shows that the uncut plasmid DNA isolated from C. hydrothermalis migrates somewhat slower than that isolated from E. coli. This may be due, at least in part, to differences in the degree of methylation of the DNA in these different hosts. These results suggest that C. hydrothermalis, like C. bescii, contains a functional CbeI/M.CbeI-like restriction-modification system (Chung et al., 2012 PLoS One 7:e43844; Chung et al., 2011 J Ind Microbiol Biotechnol 38:1867-1877). This is supported by the observation that pDCW89 isolated from C. hydrothermalis was resistant to digestion by purified CbeI or HaeIII endonucleases (FIG. 3D).

Cloning of a CBM and a Linker Region of the celA Coding Region into pDCW89.

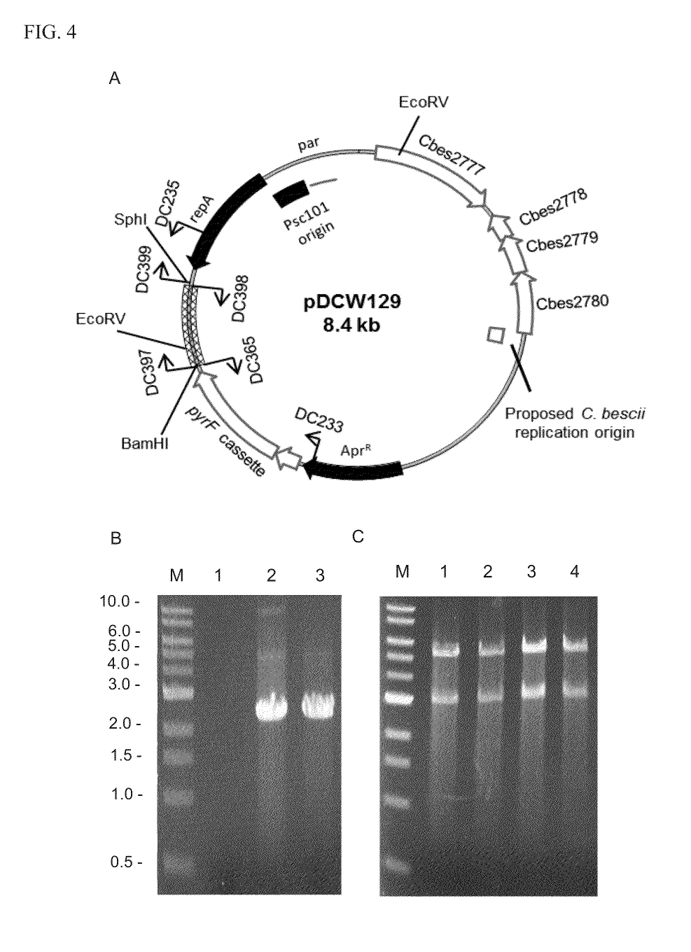

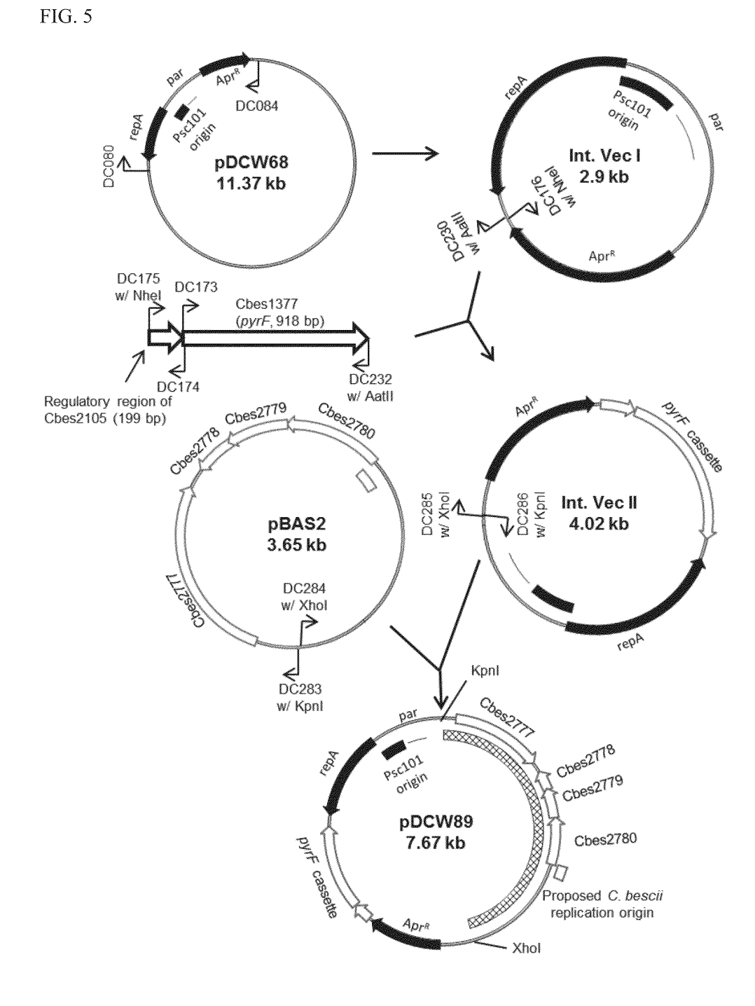

To test the use of pDCW89 as a cloning vector, a 0.68 kb DNA fragment containing a carbohydrate binding domain (CBM) and linker region derived from celA (Cbes1867) was cloned into pDCW89 (FIG. 4, pDCW129). Methylated pDCW129 was successfully transformed into JWCB005 at a comparable transformation efficiency to pDCW89. Transformation of C. bescii with pDCW129 was initially confirmed by PCR amplification of the region spanning the pyrF cassette and only in the plasmid (FIG. 4B). Total DNA isolated from JWCB018 transformants was used to “back-transform” into E. coli and plasmid DNA isolated from these back-transformants was analyzed by restriction digestion by EcoRV (FIG. 4C) and EcoRI and AatII. pDCW129 DNA isolated from the “back transformants” was indistinguishable from the pDCW129 used to transform C. bescii and showed no obvious signs of rearrangement or deletion through transformation and replication in JWCB005.

### Investigation of the Minimal Sequence Requirement for Stable Plasmid Replication.

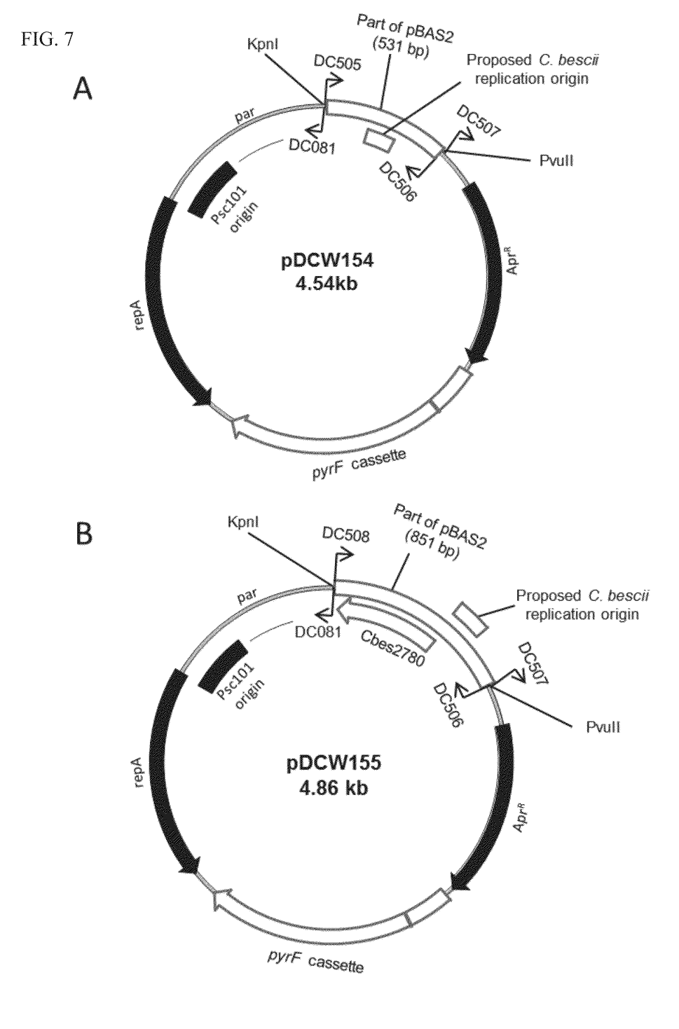

To determine the loci required for replication of shuttle vector, we constructed plasmids (pDCW154 and pDCW155, FIG. 7) that contain a portion of pBAS2. Both of these plasmids contain sequences upstream of the region of Cbes2780 that has similarity to the conserved replication nick site in double stranded origins of known rolling circle plasmids (Gruss and Ehrlich, 1989 Microbiol Rev 53:231-241; Karita et al., 2001 Biosci Biotechnol Biochem 65:226-228), in addition to 9 bp direct repeats, 21 bp direct repeats, and 134 bp of the proposed plus-stranded replication origin (Clausen et al., 2004 Plasmid 52:131-138). pDCW155 also contains the entire sequence of Cbes2780 encoding the putative rolling circle replication protein. Numerous attempts to transform these plasmids in experiments with pDCW89 as a control failed to yield transformants. There are three other ORFs in pBAS2 that may be involved in stable plasmid replication (Dam et al., 2011 Nucleic Acids Res 39:3240-3254; Clausen et al., 2004 Plasmid 52:131-138). Cbes2777 (993 bp) shows 37% sequence similarity with recombinase XerD in Thermoanaerobacterium thermosaccharolyticum M0795. Cbes2778 and Cbes2779 showed no significant similarity with known proteins. Failure to eliminate portions of pBAS2 without loss of plasmid replication suggests that most or all of the plasmid is necessary.

Thus, in one aspect, this disclosure provides a novel shuttle vector than can be used to transfer one or more heterologous polynucleotides between a Caldicellulosiruptor spp. microbe and another host microbe. Typically, the host microbe may be an amplification host—i.e., a host microbial cell that maintains the shuttle vector at a high copy number and/or can be grown to high cell density with the shuttle vector so as to amplify the shuttle vector. Generally, the shuttle vector includes an origin of replication sequence from the amplification cell, an origin of replication for Caldicellulosiruptor spp., a selectable marker for the amplification cell, and a heterologous coding sequence that complements a functional deletion in the Caldicellulosiruptor spp. genome.

In some embodiments, the amplification cell can be E. coli.

In some embodiments, the selectable marker includes a marker that can select host cells transformed with the shuttle vector from host cells that lack the shuttle vector. Exemplary selectable markers include, for example, antibiotic resistance. Exemplary antibiotics against which the shuttle vector can provide resistance include and antibiotic that works against E. coli including, for example, apramycin, spectinomycin, ampicillin, kanamycin, chloramphenicol, etc.

In some embodiments, the Caldicellulosiruptor spp. can be C. bescii. In other embodiments, however, the Caldicellulosiruptor spp. can include any member of the genus Caldicellulosiruptor such as, for example, C. hydrothermalis, C. acetigenus, C. kristjanssonii, C. kronotskiensis, C. lactoaceticus, C. obsidiansis, C. owensensis, or C. saccharolyticus.

As used herein, a “functional deletion in the Caldicellulosiruptor spp. genome” refers to any decrease in expression of a coding region that may be complemented by providing a shuttle vector that includes a heterologous polynucleotide. Thus, a “functional deletion” need not require the complete deletion of the a particular coding region and/or regulatory region controlling expression of the particular coding region.

“Complement” and variations thereof refer to the curing of a biological deficiency by providing a heterologous polynucleotide effective to cure the biological deficiency. Typically, the biological deficiency can be growth rate and/or maximum cell density when grown under certain conditions. The heterologous polynucleotide may directly correspond to a functional deletion in the genome—i.e., provide a copy of a coding region lost in the genome due to a deletion in the genome.

As used herein, “heterologous polynucleotide” and “heterologous coding region” are used interchangeably and refer to a polynucleotide that encodes a polypeptide product and that has been placed under the control of a promoter and/or other regulatory sequence that does not natively regulate expression of the heterologous polynucleotide. Thus, in some cases, a heterologous polynucleotide can encode a polypeptide product that is not natively produced by a host cell. In other cases, however, a heterologous polynucleotide can encode a polypeptide that is natively expressed by the host cell but has been placed under the regulatory control of a different promoter and/or other regulatory sequence.

In some embodiments, therefore, the shuttle vector can include a Caldicellulosiruptor spp. coding region that complements a genomic functional deletion in a Caldicellulosiruptor spp. strain. One exemplary embodiment described above involves a Caldicellulosiruptor bescii strain that involves a deletion in the pyrFA region of the genome. The shuttle vector can include a wild type (or, in some cases, a genetically modified variant of) pyrF that cures the genomic pyrF deficiency.

In some embodiments, the heterologous coding sequence of the shuttle vector can include a coding sequence from a species different than the Caldicellulosiruptor spp. In one exemplary embodiment, the shuttle vector can include one or more coding regions from another thermophilic microbe such as, for example, Clostridium thermocellum. For example, a shuttle vector can include the C. thermocellum adhE coding region, which encodes the C. thermocellum bifunctional acetaldehyde/alcohol dehydrogenase (AdhE). In other embodiments, the heterologous coding sequence can encode any wild-type or mutant form of a polypeptide that has lignocellulosic activity such as, for example, CelA, related glycosyl hydrolases, transporters that extend the breadth of substrates that may be degraded by a host cell.

As will be described in more detail below, the C. thermocellum adhE can allow C. bescii to produce ethanol directly from lignocellulosic biomass without the lignocellulosic biomass having to be pretreated. Thus, in some embodiments, the heterologous coding sequence of the shuttle vector can encode a polypeptide that is involved in biosynthesis of a biofuel or another commercially or industrially relevant chemical.

As used herein, “biosynthesis” refers to any process in which at least one step in the synthesis of a chemical compound is performed by a living organism. Thus, biosynthesis can refer to a process in which a compound is synthesized by a process that includes steps performed by a living organism and also includes steps that are performed without the involvement of a living organism—e.g., a process in which a compound is produced by a living organism, collected, and then subjected to one or more additional steps that do not involve the living organism. As used herein, a “bioproduct” is any compound that is the product of “biosynthesis.”

As used herein, the term “biofuel” refers includes, but is not limited to, a bioalcohol such as, for example, ethanol, propanol, and butanol; biodiesel; bioethers; and biogas.

In another aspect, this disclosure provides methods that involve the genetic modification of one or more organisms by transferring genetic material into an organism, or between two or more organisms, using the shuttle vectors described herein. Generally, therefore, such methods can include introducing into a cell any embodiment of the shuttle vector described above. In some embodiments, the cell can be a Caldicellulosiruptor spp. such as, for example, C. bescii. In other embodiments, the cell can be, for example, E. coli.

In yet another aspect, therefore, this disclosure provides a genetically modified cell that includes any embodiment of the shuttle vector described above.

Heterologous Expression of Clostridium thermocellum Polynucleotide in C. bescii.

We introduced a heterologous nucleotide into C. bescii using a vector similar in relevant aspects to the shuttle vector described above. As a result, we successfully metabolically engineered the C. bescii to produce ethanol from plant biomass without conventional pretreatment. This was accomplished by expressing a heterologous bi-functional acetaldehyde/alcohol dehydrogenase coding region (adhE) from Clostridium thermocellum to introduce a pathway for ethanol production. The engineered strain produced 12.8 mM ethanol directly from 2% (wt/v) switchgrass—a real world plant biomass substrate—and decreased acetate production by 38% compared to wild type. Direct conversion of lignocellulosic biomass to ethanol represents a new paradigm for consolidated bioprocessing and offers the potential for carbon neutral, cost effective, sustainable fuel production.

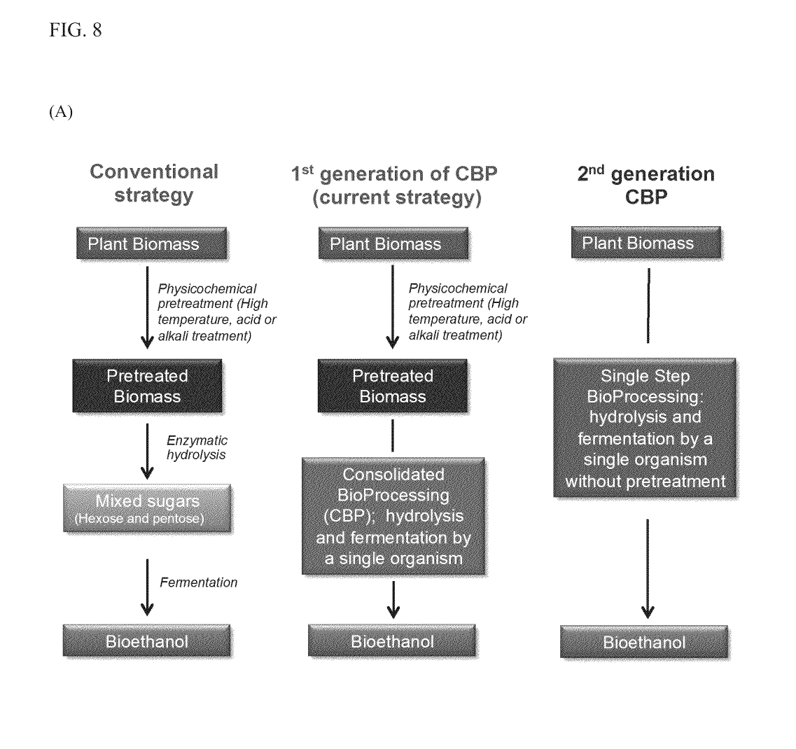

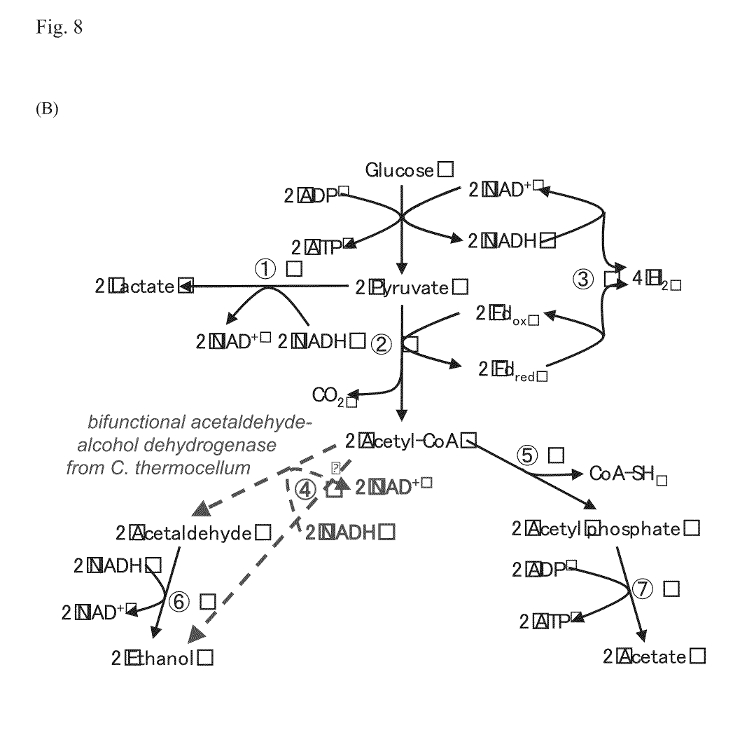

Conventional strategies for bioethanol production from lignocellulosic feed stocks typically involve physicochemical pretreatment, enzymatic saccharification, and/or fermentation (FIG. 8A) (Peralta-Yahya et al., 2012 Nature 488:320-328; Tilman et al., 2009 Science 325:270-271). Considerable effort has been made to develop single microbes capable of both saccharification and fermentation to avoid the substantial expense of using saccharolytic enzyme cocktails (FIG. 8A) (Olson et al., 2012 Curr Opin Biotechnol 23:396-405). Heterologous expression of saccharolytic enzymes has been attempted in a number of organisms—including, e.g., Saccharomnyces cerevisiae, Zymomonas mobilis, Escherichia coli, and Bacillus subtilis—to ferment various model cellulosic and hemicellulosic substrates (Olson et al., 2012 Curr Opin Biotechnol 23:396-405; Linger et al., 2010 Appl Environ Microbiol 76:6360-6369). Although these approaches have resulted in progress in cellulose utilization, the overall enzyme activity is still very low compared to that of naturally cellulolytic organisms and the rates of hydrolysis are not sufficient for an industrial process (Argyros et al., 2011 Appl Environ Microbiol 77:8288-8294).

C. bescii is the most thermophilic cellulolytic bacterium so far described, growing optimally at ˜80° C. with the ability to use, without pretreatment, a wide range of substrates such as, for example, cellulose, hemicellulose, and lignocellulosic plant biomass (Blumer-Schuette et al., 2008 Curr Opin Biotechnol 19:210-217; Yang et al., 2009 Appl Environ Microbiol 75:4762-4769). C. bescii also efficiently ferments both C₅ and C₆ sugars derived from plant biomass. The shuttle vector provided herein can simplify methods for the genetic manipulation of members of the Caldicellulosiruptor genus and opens the door for metabolic engineering for the direct conversion of plant biomass to liquid fuels such as ethanol (FIG. 8A).

C. bescii uses the Embden-Meyerhof-Parnas (EMP) pathway to convert glucose to pyruvate, which supplies various pathways that ultimately produce, for example, acetate, lactate, and hydrogen (FIG. 8B) (Yang et al., 2009 Appl Environ Microbiol 75:4762-4769). A mutant strain of C. bescii (JWCB018) was recently isolated in which the lactate dehydrogenase coding region (ldh) was disrupted spontaneously via insertion of a native transposon (Cha et al., 2013 J Ind Microbiol Biotechnol 40:1443-1448). A complete deletion of ldh was engineered (Cha et al., 2013 Biotechnol Biofuels 6:85; Brown et al., 2011 Proc Nat Acad Sci USA 108:13752-13757) and the resulting strain no longer produced lactate, instead diverting metabolic flux to additional acetate and H₂.

While many mixed acid fermentation organisms use a bifunctional acetaldehyde/alcohol dehydrogenase (AdhE) to reduce acetyl-CoA into acetaldehyde and then into ethanol, bioinformatic analysis indicates that the C. bescii genome does not encode an obvious AdhE or acetaldehyde dehydrogenase (AldH) (Carere et al., 2012 BMC Microbiology 12:295). Indeed, C. bescii does not natively produce ethanol. The phylogenetically related thermophilic Firmicute Clostridium thermocellum, however, encodes an NADH-dependent AdhE (Cthe0423) that is involved in ethanol production in C. thermocellum. Based on its known thermostability, coenzyme specificity (NADH-dependent), similarity in codon usage, and favorable catalytic stoichiometry, this adhE was a promising candidate to generate an ethanol production pathway in C. bescii. We used the ldh deletion mutant strain of C. bescii to express C. thermocellum adhE and produce ethanol from lignocellulosic biomass.

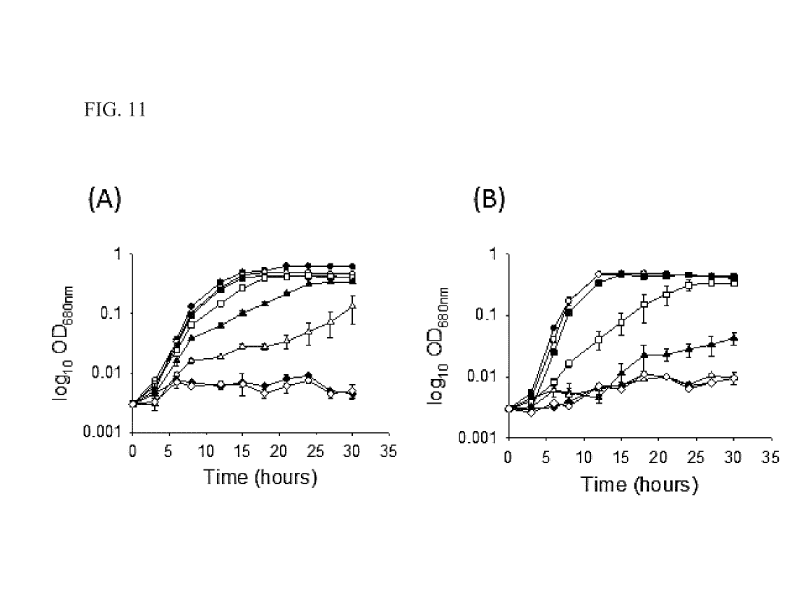

First, to determine the concentration at which growth of C. bescii is inhibited by ethanol, wild-type C. bescii was grown in LOD medium with 1% cellobiose as the sole carbon source and subjected to different levels of added ethanol at 65° C. (FIG. 11A) and 75° C. (FIG. 11B). The doubling time at 65° C. was approximately four hours and the doubling time at 75° C. was approximately 2.7 hours in the absence of ethanol. Growth of C. bescii in the presence of 300 mM added ethanol (13.8 g/l) was comparable to that with no added ethanol, reaching a maximum OD of approximately 0.6 at both 65° C. (in 15-20 hours) and 75° C. (in 10-15 hours). The addition of 400 mM ethanol (18.4 g/l) had a modest effect on growth at 65° C. (a doubling time of approximately 4.5 hours and a maximum OD of 0.44), but it substantially inhibited growth at 75° C. (a doubling time of approximately 4.0 hours and a maximum OD of 0.34). Growth was severely affected at 500 mM added ethanol (23 g/1) at 65° C. and 450 mM added ethanol (20.7 g/l) at 75° C. No growth was seen at higher ethanol concentrations (>600 mM). This level of ethanol tolerance in C. bescii is comparable to that observed in C. thermocellum (Brown et al., 2011 Proc Nat Acad Sci USA 108:13752-13757) and suggests that C. bescii can be engineered to produce ethanol at high yield.

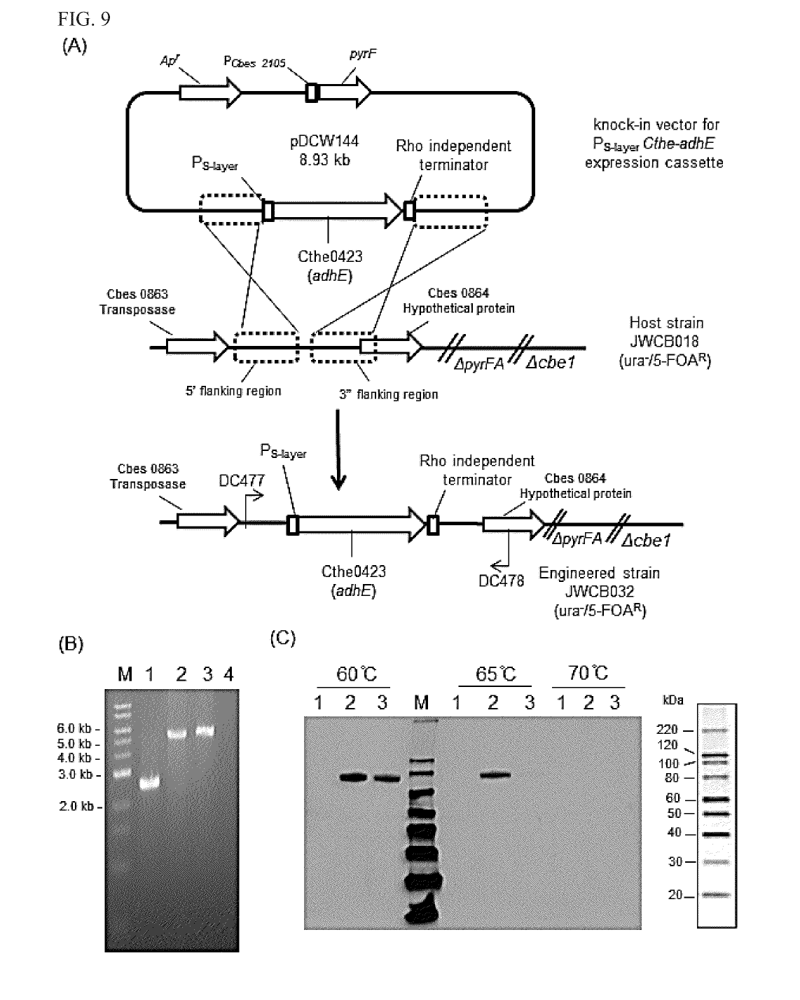

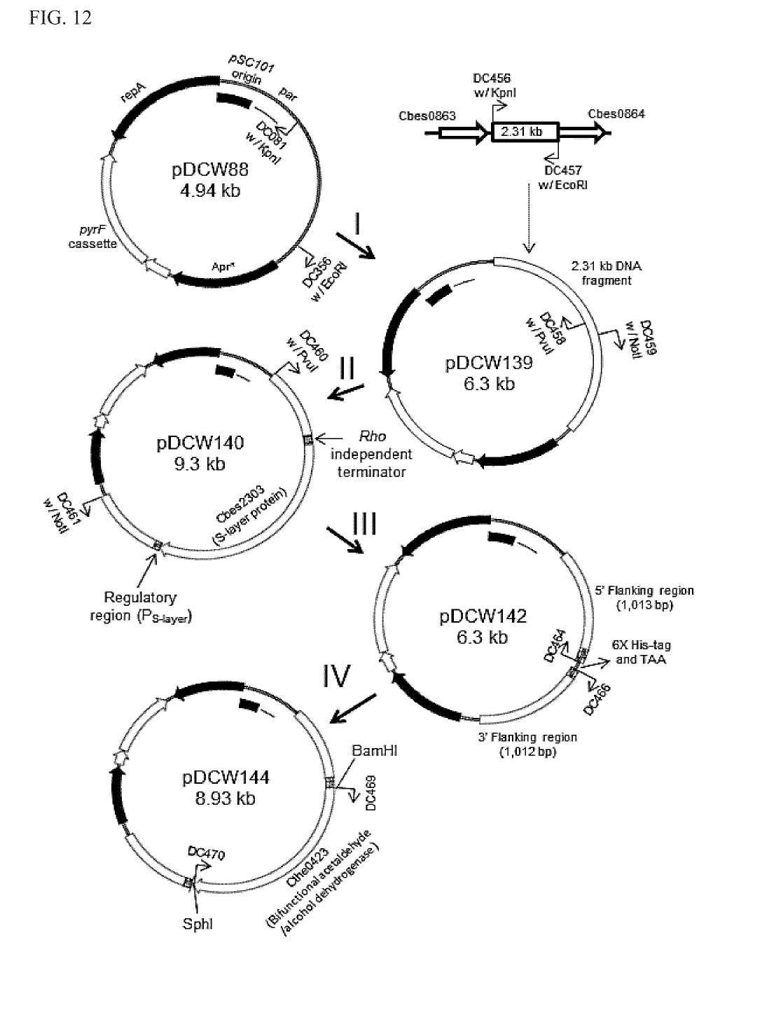

The C. thermocellum adhE coding region (Cthe0423) was amplified from C. thermocellum chromosomal DNA and cloned into pDCW144 (FIG. 12) under the transcriptional control of the C. bescii S-layer protein (Cbes2303) promoter (P_{S-layer}). RNA profiling has shown that S-layer protein RNA is abundant throughout growth, suggesting that the promoter may be strong and constitutive (Dam et al., 2011 Nucleic Acids Res 39:3240-3254). A rho-independent transcription terminator derived from a region immediately downstream of Cbes2303 was fused to the end of the adhE coding region (FIG. 9A, FIG. 12) and the vector adds a C-terminal His-tag to the AdhE protein. This P_{S-layer}-adhE construct was flanked by 2 kb DNA regions of homology from the intergenic region between Cbes0863 and Cbes0864 (FIG. 9A, FIG. 12) to allow targeted integration into the C. bescii chromosome. The resulting vector pDCW144 contains a pyrF cassette as a positive and counter-selectable marker (Chung et al., 2013 PLoS ONE 8:e62881). pDCW144 does not contain an origin of replication for C. bescii so that uracil prototrophy selection required plasmid integration into the chromosome. In all other respects, however, pDCW144 is consistent with a shuttle vector based on pDCW89 (FIG. 2A) so that expression of the C. thermocellum adhE coding region from pDCW144 can be extrapolated to shuttle vectors based on pDCW89.

The pDCW144 plasmid was transformed into the C. bescii ldh mutant strain JWCB018 (ΔpyrFA ldh::ISCbe4 ΔcbeI (Table 3), referred to herein as ldh⁻), which is a uracil auxotroph and contains a deletion of the CbeI restriction enzyme to simplify genetic manipulation (Cha et al., 2013 J Ind Microbiol Biotechnol 40:1443-1448; Chung et al., 2013 Biotechnol Biofiels 6:82). Counter-selection with 5-fluoroorotic acid (5-FOA) selected for segregation of the merodiploid as previously described (Chung et al., 2013 Biotechnol Biofuels 6:82), depicted in FIG. 9A. Two of the forty transformants analyzed by PCR amplification using primers DC477 and DC478 (FIG. 9B) contained segregated insertions of the P_{S-layer}-adhE cassette at the targeted chromosome site, resulting in strain JWCB032 (ΔpyrFA ldh::ISCbe4 ΔcbeI P_{S-layer}-adhE⁺; referred to herein as ldh⁻ adhE⁺). As shown in FIG. 9B, the parent strain, JWCB018, produced the expected wild type band at approximately 2.44 kb (lane 1), while amplification from JWCB032 (ldh⁻ adhE⁺) produced a band at approximately 5.04 kb (lane 2), indicating a knock-in of the expression cassette within this region.

Heterologous expression of the AdhE protein was detected in transformants containing the expression cassette by Western hybridization using monoclonal antibodies targeting the His-tag (FIG. 9C). Cells were grown to mid-log phase at 60° C., 65° C., and 70° C. (and 75° C., data not shown). Expression of the wild type AdhE protein was readily detected in cells grown at 60° C. and 65° C. but not at 70° C. Since the optimal temperature for growth for C. thermocellum is 60° C., the AdhE protein or adhE mRNA may not be stable at 70° C. All further experiments were performed at 65° C. to ensure production of AdhE and maximize the efficiency of biomass deconstruction by C. bescii. We also expressed a variant of the AdhE protein, AdhE*, from C. thermocellum EA that had been shown to increase ethanol tolerance without losing functional activity for ethanol production (Brown et al., 2011 Proc Nat Acad Sci USA 108:13752-13757). Interestingly, the AdhE* was detected at 60° C. but not 65° C. or 70° C. (FIG. 9C, lane 3), suggesting that either the protein or its mRNA is less thermostable than the wild type.

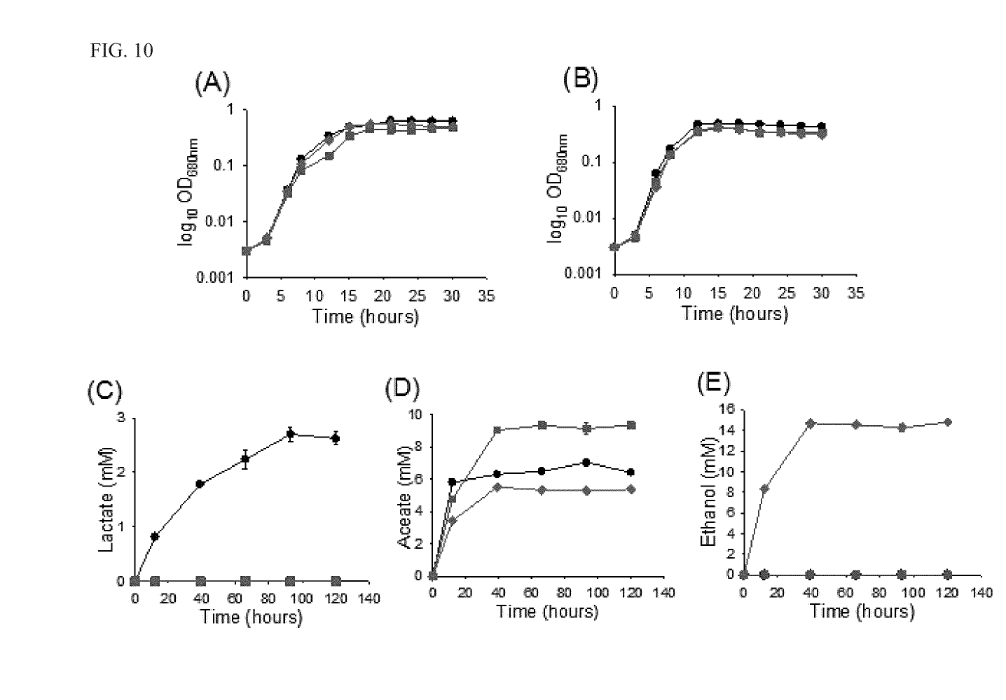

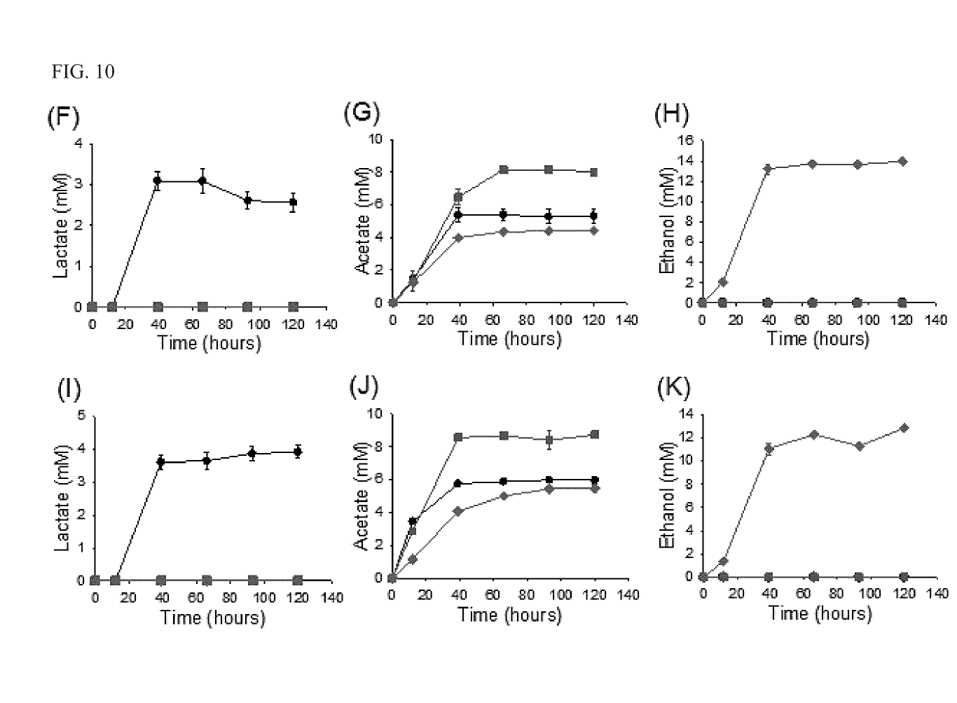

Introduction of a new fermentative pathway for ethanol production in C. bescii might have resulted in poor growth due to a redox imbalance. Therefore, we examined the growth rate and yield of the adhE-expressing strain relative to the wild type and parent strains (FIG. 10A, B). At both 65° C. and 75° C., the growth rate and growth yield were comparable between wild type, ldh⁻, and ldh⁻ adhE⁺ strains.

To determine the functionality of AdhE in C. bescii and its effect on the redirection of flux to ethanol, the fermentation product profiles from C. bescii wild type and mutant strains were examined via high performance liquid chromatography (HPLC) during growth on a soluble substrate (cellobiose, 1%), a model microcrystalline cellulosic substrate (Avicel, 2% wt/vol), and a plant biomass substrate (switchgrass, 2% wt/vol) (FIG. 10C-K). The wild type strain produces lactate (3.1 mM) and acetate (5.4 mM) but no detectable ethanol on cellobiose, avicel or switchgrass. The ldh⁻ mutant strain JWCB018 makes no detectable lactate and increased acetate (7.3˜8.2 mM) compared to the wild type, but no detectable ethanol. The ldh⁻ adhE⁺ strain, JWCB032, produced a lower level of acetate (4.3 mM) and redirected most of the flux to ethanol (14.8 mM). The level of ethanol produced on 2% Avicel (14.0 mM) was similar to that on 1% cellobiose (14.6 mM), and slightly less (12.8 mM) on switchgrass.

Surprisingly, with no adhE expression optimization or pathway manipulation aside from the use of an ldh mutant background, 70%-73% of the detected fermentation products (excluding hydrogen) in the ldh⁻ adhE⁺ strain was ethanol during growth on cellobiose, Avicel, and switchgrass. By 39 hours, cellobiose fermentation was complete. At this point, 14.7 mM ethanol had been produced from 7.4 mM cellobiose (14.8 mM glucose equivalents) that had been removed from the culture, resulting in a molar yield of 0.99 mol ethanol/mol glucose equivalents (FIG. 10).

Thus, this disclosure describes the metabolic engineering of a hyperthermophilic organism for the conversion of lignocellulosic biomass to a liquid fuel. Furthermore, ethanol has been produced directly from plant biomass without the use of harsh, expensive, or chemical pretreatment. Combining metabolic engineering with the native cellulolytic ability of C. bescii has the potential to transform the biofuels industry by creating a process in which the pretreatment step and/or the addition of exogenous cellulases may be eliminated.

The strains, vectors, and methods described herein can serve as platforms for further metabolic engineering to increase yield and titer to allow cellulosic biofuel production on an industrial scale. As approximately 25% of the carbon fermentation products remain to be redirected to ethanol, one strategy for improving yield can involve deleting or otherwise reducing the activity of one or more of the two hydrogenase coding regions and the acetate kinase coding region in C. bescii. For example, deletion of the NADH-dependent hydrogenase, pta and ack (enzyme 5 and enzyme 7 in FIG. 8, respectively) can contribute to an increase in ethanol production in T. saccharolyticum. Another strategy for increasing the ethanol yield may involve using a more thermostable heterologous AdhE protein.

The strains, vectors, and methods described herein can serve as platforms for the production of other classes of products derived from thermophilic organisms with proper folding at high temperature and physiologically relevant post-translational modifications, such as methylation and glycosylation.

Thus, in another aspect, this disclosure provides a genetically modified Caldicellulosiruptor spp. microbe engineered to increase biosynthesis of a bioproduct. Generally, the genetically modified Caldicellulosiruptor spp. microbe exhibits an increase in biosynthesis of the bioproduct compared to a wild type control.

In some cases, a wild type control may be unable to biosynthesize a particular bioproduct. In such cases, any detectable biosynthesis of the bioproduct may be considered and increases in biosynthesis of the bioproduct compared to a wild type control. In other cases, an increase in biosynthesis of a bioproduct compared to a wild type control can be quantitatively measured and described as a percentage of the biosynthetic activity of an appropriate wild-type control. The biosynthetic activity exhibited by a genetically modified microbe can be, for example, at least 110%, at least 125%, at least 150%, at least 175%, at least 200% (two-fold), at least 250%, at least 300% (three-fold), at least 400% (four-fold), at least 500% (five-fold), at least 600% (six-fold), at least 700% (seven-fold), at least 800% (eight-fold), at least 900% (nine-fold), at least 1000% (10-fold), at least 2000% (20-fold), at least 3000% (30-fold), at least 4000% (40-fold), at least 5000% (50-fold), at least 6000% (60-fold), at least 7000% (70-fold), at least 8000% (80-fold), at least 9000% (90-fold), at least 10,000% (100-fold), or at least 100,000% (1000-fold) of the activity of an appropriate wild-type control.

In some embodiments, the bioproduct can include a biofuel such as, for example, a bioalcohol such as, for example, ethanol, propanol, and butanol; biodiesel; bioethers; and biogas.

In some embodiments, the genetically modified Caldicellulosiruptor spp. microbe can exhibit the increase in biosynthesis of the bioproduct compared to a wild type control when grown on lignocellulosic biomass. As used herein, lignocellulosic biomass refers to biomass that includes carbohydrate polymers (e.g., cellulose, and/or hemicellulose) and lignin, an aromatic polymer. Lignocellulosic biomass includes materials such as, for example, switchgrass, corn stover, straw, sugarcane bagasse, woody plants such as trees, and other terrestrial plants.

In some embodiments, at least 50% of the fermentation product of the genetically modified Caldicellulosiruptor spp. microbe can be a desired bioproduct such as, for example, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the fermentation product of the genetically modified Caldicellulosiruptor spp. microbe can be a desired bioproduct.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Strains, Media and Growth Conditions

C. bescii, C. hydrothermalis, and E. coli strains are listed in Table 1. Primers are listed in Table 2.

Caldicellulosiruptor species were grown anaerobically in liquid or solid modified DSMZ516 medium (Chung et al., 2012 PLoS One 7:e43844) or in low osmolarity defined (LOD) growth medium (Farkas et al., 2012 J Ind Microbiol Biotechnol 40:41-49) with maltose as the sole carbon source as described at 75° C. for C. bescii or at 68° C. for C. hydrothermalis. For growth of auxotrophic mutants JWCB005 and JWCH003, the defined medium containing 40 μM uracil was used. E. coli strains DH5α (dam⁺ dcm⁺), BL21 (dam⁺ dcm⁺), and ET12567 (dam⁻ dcm⁻) were used for plasmid DNA constructions and preparations. Standard techniques for E. coli were performed as described (Sambrook et al., Molecular Cloning: A Laboratory Manual; Cold Spring Harbor Laboratory Press; 2001). E. coli cells were grown in L broth supplemented with apramycin (50 μg/ml), kanamycin (25 μg/ml), or spectinomycin (20 μg/mL), where appropriate. E. coli plasmid DNA was isolated using a miniprep kit (Qiagen, Valencia, Calif.). Chromosomal DNA from Caldicellulosiruptor species was extracted using the Quick-gDNA™ MiniPrep (Zymo Research Corp., Irvine, Calif.) according to the manufacturer's instructions. Total DNA was isolated from Caldicellulosiruptor species as described (Lipscomb et al., 2011 Appl Environ Microbiol 77:2232-2238), except that adding additional lysozyme (30 μg/ml) for 1 hour at room temperature in lysis buffer (Chung et al., 2011 J Ind Microbiol Biotechnol 38:1867-1877) and sonication were employed to enhance the cell lysis. Plasmid DNA isolation from Caldicellulosiruptor species was performed as described (Chung et al., 2011 J Ind Microbiol Biotechnol 38:1867-1877).

### Isolation of 5-FOA Resistant/Uracil Auxotrophic Mutants

A spontaneous deletion within the C. bescii DSM6725 pyrFA locus (FIG. 1A, Table 1) was isolated using the same approaches as previously described (Chung et al., 2012 PLoS One 7:e43844). Growth of this strain. JWCB005, supplemented with uracil (40 μM) was comparable to wild type reaching a cell density of ˜2×10⁸ in 24 hours. Cells were counted in a Petroff-Housser counting chamber using a phase-contrast microscope with 40× magnification.

### Construction of Plasmids

Plasmids were generated using high fidelity pfu AD DNA polymerase (Agilent Technologies, Inc., Santa Clara, Calif.), restriction enzymes (New England Biolabs, Inc., Ipswich, Mass.), and Fast-link™ DNA Ligase (Epicentre Bioechnologies Corp., Madison, Wis.) according to the manufacturer's instructions. Plasmid pDCW89 (FIG. 2A, FIG. 5) was constructed in three cloning steps. First, a 2.9 kb of DNA fragment, containing the PSC 101 replication origin and an apramycin resistance cassette (Apr^{R}), was amplified by PCR using the primers DC080 and DC084 and pDCW68 as template (Chung et al., 2011 J Ind Microbiol Biotechnol 38:1867-1877) as template. The 2.9 kb DNA fragment was then blunt-end ligated after treatment with T4 PNK (New England Biolabs, Inc., Ipswich, Mass.) to construct Intermediate vector I (FIG. 5). A cassette containing the wild-type pyrF coding region was constructed by overlap extension polymerase chain reaction (OE-PCR) placing the pyrF coding region under the transcriptional control of the Cbes2105 (30S ribosomal protein S30EA) promoter. A 199 bp portion of the regulatory region of Cbes2105 was amplified from wild-type genomic DNA using primers DC 175 and DC 174. The pyrF (Cbes1377, 918 bp) coding region was amplified using primers DC 173 and DC232, and joined to the fragment containing the regulatory region by OE-PCR. The DC 175 and DC232 primers were engineered to contain NheI and AatII sites, respectively. The 2.9 kb DNA fragment were amplified by PCR from intermediate vector 1 using primers DC 176 and DC230 to add restriction sites. The DC176 and DC230 primers were also engineered to contain NheI and AatII sites, respectively. The two linear DNA fragments were digested with NheI and AatII, and ligated to generate 4.02 kb size of intermediate vector II (FIG. 5). In the last step, a 3.65 kb DNA fragment containing the entire sequence of pBAS2 (Dam et al., 2011 Nucleic Acids Res 39:3240-3254; Clausen et al., 2004 Plasmid 52:131-138) were amplified by PCR using DC283 and DC284, that contained restriction sites added a KpnI site at 5′ end and a XhoI site at 3′ end. A 4.02 kb linear fragment was amplified from intermediate vector II using DC285 and DC286, which contain engineered restriction sites, KpnI and XhoI, respectively. The two linear DNA fragments were digested with KpnI and XhoI, and ligated to generate pDCW89. Further details of this construction are described in FIG. 5. Plasmid pDCW129 (FIG. 4A) was generated inserting a 0.68 kb DNA fragment containing the carbohydrate binding domain (CBM) and linker region derived from celA (Cbes1867) into pDCW89. A 0.68 kb DNA fragment was amplified by PCR using the DC397 and DC398 primers and total DNA isolated from C. bescii as template. The 7.75 kb of backbone DNA fragment was amplified by PCR using the DC365 and DC399 and pDCW89 as template. DC397 and DC365 primers were engineered to contain a BamHI site at the end. DC398 and DC399 primers were engineered to contain an SphI site at the end. The two linear DNA fragments were digested with BamHI and SphI and ligated to generate pDCW129. Plasmid pDCW154 and pDCW155 were generated to reduce the size of pDCW89 (FIG. 7). The 4.02 kb backbone DNA fragment was amplified by PCR using primers DC081 and DC507 and pDCW89 as template. The DC081 and DC507 primers were engineered to contain KpnI and PvuII sites, respectively. To generate pDCW154, a 531 bp DNA fragment derived from pBAS2 was amplified by PCR using DC505 and DC506, primers with engineered restriction sites, KpnI and PvuII, respectively. Two linear DNA fragments were digested with KpnI and PvuII, and ligated to generate pDCW154 (FIG. 7A). Plasmid pDCW155 (FIG. 7B) is identical to pDCW154 except that the 851 bp DNA fragment was replaced with the 531 bp of fragment. The 851 bp DNA fragment derived from pBAS2 was amplified by PCR using DC508 and DC 506, primers with engineered restriction sites, KpnI and PvuII, respectively. This 851 bp DNA fragment was subsequently ligated into a 4.02 kb backbone DNA fragment as described for pDCW154. DNA sequences of the primers are shown in Table 2. E. coli strain DH5α cells were transformed by electroporation in a 2-mm-gap cuvette at 2.5 V and transformants were selected for apramycin resistance. The sequences of all plasmids were confirmed by automatic sequencing (Macrogen Corp., Rockville, Md.).

### Transformation of Caldicellulosiruptor Species

Electrotransformation of JWCB005 and JWCH003 was performed as described (Chung et al., 2012 PLoS One 7:e43844). JWCB011 and JWCB014 were generated by transforming JWCB005 with M.CbeI methylated pDCW89 and/or pDCW129 as described and selecting for uracil prototrophy at 75° C. DNA transformation of C. bescii was confirmed by PCR analysis using primers DC230 and JF199 or primers DC233 and DC235, and also by back-transformation to E. coli. Transformation of the JWCH003 strain was performed similarly, but at 68° C. Transformation of pDCW89 into C. hydrothermalis was confirmed by direct plasmid DNA isolation from transformant, JWCH005. The transformation efficiencies were calculated as the number of transformant colonies per μg of DNA added and do not take into account plating efficiencies. E. coli strain DH5α cells were transformed by electroporation in a 2 mm gap cuvette at 2.5 V, and transformants were selected for apramycin resistance.

### Assessment of Relative Copy Number, Maintenance, and Stability

C. bescii transformants (JWCB011) were serially subcultured every 16 hours for five passages in selective (without uracil) and non-selective (supplemented with 40 M uracil) liquid media. After each passage, cells were harvested and used to isolate total DNA. For each sample, 3 μg of total DNA was digested with 10 U of EcoRV for six hours at 37° C. The restriction fragments were separated by electrophoresis in a 1.0% (wt/vol) agarose gel and transferred onto nylon membranes (Roche Diagnostics, Indianapolis, Ind.). Primers JF396 and JF397 (Table 2) were used to amplify a fragment of the pyrF coding region using JWCB005 genomic DNA as template to generate a digoxigenin (DIG)-labeled probe by random priming with DIG High Prime DNA Labeling and Detection Starter Kit I (Roche Diagnostics, Indianapolis, Ind.). The membrane was incubated with probe at 42° C. and washed at 65° C. Band intensities were determined by using a Storm 840 PhosphorImager (Molecular Dynamics, Inc., Sunnyvale, Calif.) equipped with ImageQuaNT™ v.5.4 software (GE Healthcare, Little Chalfont, United Kingdom). Relative copy number was determined as the ratio of band intensity of the plasmid-derived band to the chromosomal pyrF fragment. Plasmid maintenance with and without selection was inferred from the change in relative copy number over the five successive cultures. To assess the structural stability of the plasmid, total DNA isolated from five independent C. bescii transformants containing pDCW89 was used to back-transform E. coli for plasmid isolation and restriction digestion analysis.

Determining the Relative Copy Number of pBAS2

Total DNA was isolated from JWCB001 and treated with RNase A (Qiagen, Valencia, Calif.). qPCR experiments were carried out with an LightCycler 480 Real-Time PCR instrument (Roche Diagnostics, Indianapolis, Ind.) with the LightCycler 480 SYBR Green I master mix (Roche Diagnostics, Indianapolis, Ind.). The relative copy number of pBAS 2 (Dam et al., 2011 Nucleic Acids Res 39:3240-3254; Clausen et al., 2004 Plasmid 52:131-138) was determined as the average of two biologically independent samples. Table 2 lists the primers used in the qPCR experiment.

TABLE 1
Strains and plasmids

| Strains or plasmid | Strain and genotype/phenotype | Source |
|---|---|---|
| Caldicellulosiruptor | | |
| JWCB001 | C. bescii DSM6725 wild type (ura⁺/5-FOA^{S}) | DSMZ¹ |
| JWCB005 | C. bescii ΔpyrFA (ura⁻/5-FOA^{R}) | This study |
| JWCB011 | C. bescii JWCB005 transformed with pDCW89 (ura⁺/5-FOA^{S}) | This study |
| JWCB014 | C. bescii JWCB005 transformed with pDCW129 (ura⁺/5-FOA^{S}) | This study |
| JWCH003 | C. hydrothermalis ISCahyI insertion mutation in pyrF (ura⁻/5-FOA^{R}) | ^{a} |
| JWCH005 | JWCH003 transformed with pDCW89 (ura⁺/5-FOA^{S}) | This study |
| Escherichia coli | | |
| JW261 | DH5α containing pDCW68 (Apramycin^{R}) | ^{b} |
| JW292 | DH5α containing pDCW89 (Apramycin^{R}) | This study |
| JW301 | DH5α containing pDCW129 (Apramycin^{R}) | This study |
| JW319 | DH5α containing pDCW154 (Apramycin^{R}) | This study |
| JW320 | DH5α containing pDCW155 (Apramycin^{R}) | This study |
| Plasmids | | |
| pDCW68 | 6-8 copy plasmid DNA (Apramycin^{R}) | ^{b} |
| pDCW89 | E. coli/Caldicellulosiruptor species shuttle vector (Apramycin^{R}) | This study |
| pDCW129 | E. coli/Caldicellulosiruptor species shuttle vector (Apramycin^{R}) | This study |
| pDCW154 | 6-8 copy plasmid DNA (Apramycin^{R}) | This study |
| pDCW155 | 6-8 copy plasmid DNA (Apramycin^{R}) | This study |

¹German Collection of Microorganisms and Cell Culture
^{a} Chung et al., 2013 J Ind Microbiol Biotechnol 40: 517-521.
^{b} Chung et al., 2011 J Ind Microbiol Biotechnol 38: 1867-1877.

TABLE 2

| | | SEQ | |
|---|---|---|---|
| | | ID | |
| Primers | Sequences (5′ to 3′) | No. | Description |
| FJ298 Forward | ACCAGCCTAA CTTCGATCAT TGGA | 1 | To amplify pyrF (Cbes1377) region |
| JH020 Reverse | TCTGACGCTC AGTGGAACGA A | 2 | To amplify pyrE (Cbes1377) region |
| DC326 Forward | TCTGCTAGCT CAGGTCCTGC TATAAAGCCA A | 3 | To amplify pyrE (Cbes1382) region |
| DC331 Reverse | TCACACGTAC CAGAAGGCAG AC | 4 | To amplify pyrE (Cbes1382) region |
| JF199 Reverse | CGCTAACGGA TTCACCACT | 5 | To amplify pSC101 E. coli |
| | | | replication origin |
| DC080 Forward | TCATCTGTGC ATATGGACAG | 6 | To amplify E. coli features in |
| | | | pDCW68 |
| DC084 Reverse | TCCAACGTCA TCTCGTTCTC | 7 | To amplify E. coli features in |
| | | | pDCW68 |
| DC230 Forward | AAGAGACGTC TCATCTGTGC ATATGGACAG | 8 | To construct intermediate vector II |
| DC176 Reverse | TCTGCTAGCT CCAACGTCAT CTCGTTCTC | 9 | To construct intermediate vector II |
| DC175 Forward | AGAGCTAGCT TCAACAACCA GAGACACTTG GGA | 10 | To amplify pyrF cassette |
| DC174 Reverse | AGCCTATCAG AGAAGTTCAA CAATCTAGAG | 11 | To amplify the pyrE cassette |
| | ACCATCCTTT CTATGTAGAA A | | |
| DC173 Forward | TTTCTACATA GAAAGGATGG TCTCTAGATT | 12 | To amplify the pyrE cassette |
| | GTTGAACTTC TCTGATAGGC T | | |
| DC232 Reverse | AGAGACGTCT TAAGAGATTG CTGCGTTGAT A | 13 | To amplify pyrF cassette |
| DC283 Forward | TCTGGTACCA CCGTGAGCAT TCTGGACAGG T | 14 | To amplify entire pBAS2 |
| DC284 Reverse | AGACTCGAGA TTCCCATGAG CCCACGAACA GT | 15 | To amplify entire pBAS2 |
| DC285 Forward | AGACTCGAGT CTTCTGACGC TCAGTGGAAC GAA | 16 | To construct pDCW89 |
| DC286 Reverse | TCTGGTACCA CCAGCCTAAC TTCGATCATT GGAC | 17 | To construct pDCW89 |
| DC399 Forward | AGAGCATGCG AAAACTTGTA TTTCCAGGGC | 18 | To construct pDCW129 |
| | CATCACCATC ACCATCACTA ATTTCC | | |
| DC365 Reverse | TCTGGATCCA ATCCTCCTTT TGGGATTATA | 19 | To construct pDCW129 |
| | ACTGTCCATA TGCACAGATG AGACG | | |
| DC397 Forward | AGAGGATCCA TGCAGATAAA GGTATTGTAT | 20 | To construct pDCW129 |
| | GCTAAC AAG | | |
| DC398 Reverse | AGACAGAGGT TTATGTGGTT ATGGGCATGC | 21 | To construct pDCW129 |
| DC505 Forward | TCTGGTACCT CTTTATCTTC CATTATGAGT | 22 | To construct pDCW154 |
| | TTGATAG | | |
| DC506 Reverse | ACGTTAGTCA GCTG TTGTTA GTTC | 23 | To construct pDCW154 and 155 |
| DC507 Forward | AGAAGAACAG CTGTCTGACG CTCAGTGGAA CGAA | 24 | To construct pDCW154 and 155 |
| DC081 Reverse | AGAGGTACCA CCAGCCTAAC TTCGATCATT GGA | 25 | To construct pDCW154 and 155 |
| DC508 Forward | TCTGGTACCA GTTCCTGCTT TGTTAACATT CCTTG | 26 | To construct pDCW155 |
| JF396 Forward | AGTGTTCTTA TAGCTGGAAT TGCTACGAG | 27 | To produce probe within pyrF for |
| | | | southern analysis |
| JF397 Reverse | AGCGTTTGAG TATCCTTTTG CAG | 28 | To produce the probe within pyrF |
| | | | for southern analysis |
| DC233 Forward | ATCCGTTGAT CTTCCTGCAT | 29 | To confirm the transformation |
| | | | of pDCW129 |
| DC255 Reverse | AGGATCTGAG GTTCTTATGG CTC | 30 | To confirm the transformation |
| | | | of pDCW129 |
| Q1 Forward | TGGGAAAGCC GTCCATAATC | 31 | qPCR primer for pBAS2 |
| Q2 Reverse | TCTCCCGCTC TTCTCTCTTT | 32 | qPCR primer for pBAS2 |
| Q3 Forward | GTGCGTCTAC AGGACCTTAT TT | 33 | qPCR primer for pBAS2 |
| Q4 Reverse | GGCAAGATTC TACAGGCAAG A | 34 | qPCR primer for pBAS2 |
| Q5 Forward | TGAGCGCCAA TCAGGTATAA G | 35 | qPCR primer for chromosome |
| | | | (2249000-2249090) |
| Q6 Reverse | GGAAGGGAGA TAGCGGATAG A | 36 | qPCR primer for chromosome |
| | | | (2249000-2249090) |
| Q7 Forward | GCATCTGGTG GCTATGGATA TT | 37 | qPCR primer for chromosome |
| | | | (1303164-1303201) |
| Q8 Reverse | ACCTTTGCTC CACACCTTAC | 38 | qPCR primer for chromosome |
| | | | (1303164-1303201) |

### Example 2

### Strains, Media and Culture Conditions

C. bescii strains and plasmids are listed in Table 3. Primers are listed in Table 4.

All C. bescii strains were grown anaerobically in liquid or on solid surface in low osmolarity defined (LOD) medium (Farkas et al., 2013 J Ind Microbiol Biotechnol 40:41-49), final pH 7.0, with maltose (0.5%/wt/v; Sigma-Aldrich, St. Louis, Mo.) as the carbon source unless otherwise noted. Liquid cultures were grown from a 0.5% inoculum or a single colony and incubated at 75° C. in anaerobic culture bottles degassed with five cycles of vacuum and argon. For growth of uracil auxotrophic mutants, the LOD medium was supplemented with 40 μM uracil. E. coli strain DH5α was used for plasmid DNA constructions and preparations. Standard techniques for E. coli were performed as described (Sambrook et al., Molecular Cloning: A Laboratory Manual; Cold Spring Harbor Laboratory Press; 2001). E. coli cells were grown in LB broth supplemented with apramycin (50 μg/mL) and plasmid DNA was isolated using a miniprep kit (Qiagen, Valencia, Calif.). Chromosomal DNA from C. bescii strains was extracted using the Quick-gDNA™ MiniPrep (Zymo Research Corp., Irvine, Calif.) or using the DNeasy Blood & Tissue Kit (Qiagen, Valencia, Calif.) according to the manufacturer's instructions.

TABLE 3
Strains and plasmids

| | Strain and genotype/phenotype | Source |
|---|---|---|
| Strains | | |
| JWCB001 | C. bescii DSMZ6725 wild type/(ura⁺/5-FOA^{S}) | DSMZ¹ |
| JWCB018 | ΔpyrFA ldh::ISCbe4 Δcbe1/(ura⁻/5-FOA^{R}) | ^{4, 5} |
| JWCB032 | ΔpyrFA ldh::ISCbe4 Δcbe1::P_{S-layer} Cthe-adhE²/ | This study |
| | (ura⁻/5-FOA^{R}) | |
| JWCB033 | ΔpyrFA ldh::ISCbe4 Δcbe1::P_{S-layer} | This study |
| | Cthe-adhE*(EA)³/(ura⁻/5-FOA^{R}) | |
| Plasmids | | |
| pDCW88 | cbe1 deletion vector (Apramycin^{R}) | ⁴ |
| pDCW139 | Intermediate vector 1 (Apramycin^{R}) | This study |
| pDCW140 | Intermediate vector 2 (Apramycin^{R}) | This study |
| pDCW142 | Intermediate vector 3 (Apramycin^{R}) | This study |
| pDCW144 | Integrational vector containing the P_{S-layer} | This study |
| | Cthe-adhE² expression cassette | |
| pDCW145 | Integrational vector containing the P_{S-layer} | This study |
| | Cthe-adhE*³ expression cassette | |

¹German Collection of Microorganisms and Cell Cultures
²Cthe-adhE (Cthe0423; Bifunctional acetaldehyde-CoA/alcohol dehydrogenase derived from Clostridium thermocellum ATCC27405)
³Cthe-adhE*(EA) (Cthe0423-P704L, H734R: Bifunctional ace(aldehyde-CoA/alcohol debydrogenase derived from ethanol tolerant Clostridium thermocellum strain EA) (Brown et al., 2011 Proc Natl Acad Sci USA 108: 13752-13757).
⁴ Chung et al., 2013 Biotechnol Biofuels 6: 82.
⁵ Cha et al., 2013 J Ind Microbiol Biotechnol 40: 1443-1448.

TABLE 4

| | | SEQ | |
|---|---|---|---|
| | | ID | |
| Primers | Sequences (5′ to 3′) | No. | Description |
| DC081 | TCCAATGATC GAAGTTAGGC TGGT | 39 | To construct pDCW139 |
| DC356 | TCTGAATTCT CTGACGCTCA GTGGAACGAA | 40 | To construct pDCW139 |
| DC456 | AGAGGTACCT GTGAGGGCAT GTCAATTTAC GA | 41 | To construct pDCW139 |
| DC457 | AGAGAATTCT CTTTTCGATG GAATCTTCTT CGGA | 42 | To construct pDCW139 |
| DC458 | AGAGAGCGAT CGTCTATTGT AACTTTCACT TCAGTGCA | 43 | To construct pDCW140 |
| DC459 | AGAAGAAGGC GGCCGCTGGA AGAACTTGAA AGCAGGCT | 44 | To construct pDCW140 |
| DC460 | AGAGAGCGAT CGACAGTTTG ATTACAGTTT | 45 | To construct pDCW140 |
| | AGTCAGAGCT | | |
| DC461 | AGAAGAAGGC GGCCGCTTGG TTCCTTAAAT | 46 | To construct pDCW140 |
| | CTAAGAGGTA TGA | | |
| DC462 | TGCTGGCAGA GAAGAGCGAA A | 47 | Sequencing primer for pDCW140 |
| DC463 | TCTTCATCCC AATCTTCAAC TTC | 48 | Sequencing primer for pDCW140 |
| DC464 | ACTGGATCCC TCACCAAACC TCCTTGTATG AT | 49 | To construct pDCW142 |
| DC466 | AGAGCATGCC ATCACCATCA CCATCACTAA | 50 | To construct pDCW142 |
| | TAATAAAGCT GAAATAAAAG AGGGTGAGA | | |
| DC469 | ACTGGATCCA TGACGAAAAT AGCGAATAAA TACGAAGT | 51 | To construct pDCW144 and 145 |
| DC470 | AGAGCATGCT TTCTTCGCAC CTCCGTAATA | 52 | To construct pDCW144 and 145 |
| | AGCGTTCAGA | | |
| DC471 | TGGTAATGAG AGAAGCAGAT G | 53 | Sequencing primer for pDCW144 |
| | | | and 145 |
| DC472 | TGATAAAAAG CACCCAGTTT GT | 54 | Sequencing primer for pDCW144 |
| | | | and 145 |
| DC477 | TGGTTGACCA GGAGAATTTT ACACA | 55 | Sequencing primer to verify |
| | | | the insertion |
| DC478 | AGCAACAATC CTGCATTTGT AAG | 56 | Sequencing primer to verify |
| | | | the insertion |

Construction of Vectors for Knock-in of the Cthe0423 and its Derivative into C. bescii

Plasmid pDCW144 (FIG. 9A) was constructed as illustrated in FIG. 12. The plasmids described below were generated using high fidelity pfu AD DNA polymerase (Agilent Technologies, Inc., Santa Clara, Calif.) for PCR reactions, restriction enzymes (New England Biolabs Inc., Ipswich, Mass.), and Fast-link DNA Ligase kit (Epicentre Biotechnologies Corp., Madison. WI) according to the manufacturer's instructions.

A 2.31 kb DNA fragment containing the targeted insertion region sequences (intergenic space between convergent genes Cbes0863-Cbes0864) in C. bescii chromosome was amplified using primers DC456 (with KpnI site) and DC457 (with EcoRI site) using C. bescii genomic DNA as a template. The 4.0 kb DNA fragments containing an apramycin resistance cassette, pyrF cassette (Chung et al., 2013 PLoS One 8:e62881), and the pSC101 replication origin, were amplified from pDCW88 (Chung et al., 2013 Biotechnol Biofuels 6:82) using primers DC081 and DC356. The DC081 and DC356 primers were engineered to contain KpnI and EcoRI sites, respectively. These two linear DNA fragments were digested with KpnI and EcoRI, and ligated to construct 6.33 kb size of pDCW139 (FIG. 12, step I).

Plasmid pDCW140 was constructed by inserting the 3.28 kb of DNA fragment, which contains the 134 bp of upstream sequences of Cbes2303 (S-layer protein), 3,507 bp of Cbes2303 coding sequences, and 86 bp of its downstream sequences, into the pDCW139. (FIG. 12, step II) The DNA fragment was amplified using primers DC460 (with PvuI site) and DC461 (with NotI site) using C. bescii genomic DNA as a template. The 6.1 kb DNA fragment was amplified from pDCW139 using primers DC458 (with PvuI site) and DC459 (with NotI site) to be used as a back-bone fragment. These two linear DNA fragments were digested with PvuI and NotI, and ligated to construct pDCW140 (9.3 kb), which contains the 5′ flanking region (1,013 bp) and the 3′ flanking region (1,012 bp) of targeted insertion site in C. bescii genome in addition to S-layer protein expression cassette (FIG. 12).

Plasmid pDCW142 was constructed by (a) adding restriction sites for cloning and C-terminal 6× Histidine-tag in front of the stop codon and (b) removing the Cbes2303 coding sequences in pDCW140. The 6.3 kb DNA fragment was amplified from pDCW139 using primers DC464 (with BamHI site) and DC466 (with SphI site, 6× Histidine-tag, and stop codon) using pDCW140 as a template. This DNA fragment was blunt-end ligated after treatment with T4 PNK (New England Biolabs Inc., Ipswich, Mass.) to construct pDCW142 (FIG. 12, step III).

To complete construction of pDCW144, a 2.62 kb DNA fragment containing the coding sequence of Cthe0423 was amplified by PCR using DC469 (with BamHI site) and DC470 (with SphI site) using Clostridium thermocellum ATCC 27405 genomic DNA as a template. This DNA fragment was digested with BamHI and SphI, and then cloned into pDCW142 that had been digested with BamHI and SphI (FIG. 12).

Plasmid pDCW145 is identical to pDCW144 except for the cloning of Cthe0423 adhE* (EA) (Brown et al., 2011 Proc Nat Acad Sci USA 108, 13752-13757), which contains two point mutations in coding sequences, into pDCW142. To make this change, a 2.62 kb DNA fragment containing the coding sequence of Cthe0423* were amplified by PCR using DC469 (with BamHI site) and DC470 (with SphI site) using Clostridium thermocellum EtOH (Brown et al., 2011 Proc Nat Acad Sci USA 108, 13752-13757), genomic DNA as a template. E. coli strain DH5α cells were transformed by electroporation in a 2-mm-gap cuvette at 2.5 V and transformants were selected for apramycin resistance. The sequences of all plasmids were verified by automatic sequencing (Macrogen USA, Rockville, Md.).

Transformation, Screening, Purification, and Sequence Verification of Engineered C. bescii Mutants

To construct strain JWCB032, one microgram of pDCW144 DNA was used to electrotransform JWCB018 (ΔpyrFA ΔcbeI) as described (Chung et al., 2013 Biotechnol Biofiuels 6:82). Cells were then plated onto solid LOD medium and uracil prototrophic transformant colonies were inoculated into liquid medium for genomic DNA extraction and subsequent PCR screening of the targeted region to confirm the knock-in event of pDCW144 into the chromosome. Confirmed transformants were inoculated into nonselective liquid defined medium, with 40 μM uracil, and incubated overnight at 75° C. to allow loop-out of the plasmid DNA leaving the adhE expression cassette (FIG. 2A and FIG. 12). The cultures were then plated onto 5-FOA (8 mM)-containing solid medium. After initial screening, transformants containing the expected knock-in were further purified by one additional passage on solid medium and screened a second time by PCR to check for segregation of the P_{S-layer}-adhE insertion. The location of the insertion was verified by PCR amplification and sequence analysis. A PCR product was generated from genomic DNA using primers (DC477 and DC478) outside the homologous regions used to construct the knock-in, and internal primers (DC456, DC457, DC462 and DC463). PCR products were sequenced to confirm. Construction of JWCB033 was the same as JWCB032 except that pDCW145 (Table 3) was used to electrotransform JWCB018.

### Preparation of Cell Lysates and Western Blotting

Cell-free extracts of C. bescii were prepared from 500 ml cultures grown to mid-log phase at various temperatures (60° C., 65° C., 70° C., and 75° C.), harvested by centrifugation at 6,000×g at 4° C. for 15 minutes and resuspended in Cel-Lytic B cell lysis reagent (Sigma-Aldrich, St. Louis, Mo.). Cells were lysed by a combination of 4× freeze-thawing and sonication on ice. Protein concentrations were determined using the Bio-Rad protein assay kit with bovine serum albumin (BSA) as the standard. 77 microgram protein samples were electrophoresed in a 4-15% gradient Mini-Protean TGX gels (Bio-Rad Laboratories, Inc., Hercules, Calif.) and electrotransferred to PVDF membranes (IMMOBILON-P; EMD Millipore Corp., Billerica, Mass.) using a MINI-PROTEAN 3 electrophoretic apparatus (Bio-Rad Laboratories, Inc., Hercules, Calif.). The membranes were then probed with His-tag (6×His) monoclonal antibody (1:5000 dilution; Invitrogen, Life Technologies Corp., Grand Island, N.Y.) using the ECL Western Blotting substrate Kit (Thermo Scientific, Waltham, Mass.) as specified by the manufacturer.

### Growth Curve Analysis, Measurement of Ethanol Tolerance, and Fermentation Conditions

Analysis of growth and ethanol tolerance was conducted in stoppered 125 ml serum bottles containing 50 ml LOD medium supplemented with 10 g/l cellobiose (Sigma-Aldrich, St. Louis, Mo.) and 1 mM uracil. Duplicate bottles were inoculated with a fresh 2% (v/v) inoculum and incubated at both 65° C. and 75° C. with shaking at 150 rpm. Optical cell density was monitored using a Genova spectrophotometer (Jenway, Bibby Scientific, Burlington, N.J.), measuring absorbance at 680 nm. Batch fermentations were performed for five days, at 65° C. in the same culture conditions except using 10 g/l cellobiose, 20 g/l avicel (Fluka, Sigma-Aldrich, St. Louis, Mo.), or 10 g/l unpretreated (sieved −20/+80-mesh fraction; washed with warm water but no additional pretreatment) switchgrass as carbon sources.

### Analytical Techniques for Determining Fermentation End Products

Fermentation products acetate, lactate and ethanol, were analyzed on an Agilent 1200 infinity high-performance liquid chromatography (HPLC) system (Agilent technologies, Santa Clara, Calif.). Metabolites were separated on an Aminex HPX-87H column (Bio-Rad Laboratories, Hercules, Calif.) under isocratic temperature (50° C.) and a flow (0.6 ml/min) condition in 5.0 mM H₂SO₄ and then passed through a refractive index (RI) detector (Agilent 1200 Infinity Refractive Index Detector). Identification was performed by comparison of retention times with standards, and total peak areas were integrated and compared against peak areas and retention times of known standards for each interest.

The complete disclosure of all patents, patent applications, and publications, and electronically available material (including, for instance, nucleotide sequence submissions in. e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A shuttle vector for transferring genetic material between Caldicellulosiruptor spp. and an amplification cell, the shuttle vector comprising:
an origin of replication sequence from the amplification cell;
an origin of replication for Caldicellulosiruptor spp.;
a selectable marker for the amplification cell; and
a heterologous coding sequence that complements a functional deletion in the Caldicellulosiruptor spp. genome.

2. The shuttle vector of claim 1 wherein the amplification cell is E. coli.

3. The shuttle vector of claim 1 wherein the selectable marker comprises antibiotic resistance.

4. The shuttle vector of claim 3 wherein the antibiotic resistance comprises resistance to apramycin.

5. The shuttle vector of claim 1 wherein the Caldicellulosiruptor spp. is C. bescii.

6. The shuttle vector of claim 1 wherein the heterologous coding sequence comprises pyrF operably linked to a regulatory sequence.

7. The shuttle vector of claim 1 wherein the heterologous coding sequence comprises a coding region from a species different than the Caldicellulosiruptor spp.

8. The shuttle vector of claim 7 wherein the heterologous coding sequence encodes a polypeptide involved in plant biomass deconstruction.

9. The shuttle vector of claim 7 wherein the heterologous coding sequence encodes a polypeptide involved in biosynthesis of a biofuel.

10. The shuttle vector of claim 7 wherein the heterologous coding sequence encodes a polypeptide involved in biosynthesis of a bioproduct.

11. A cell comprising the shuttle vector of claim 1.

12. A method comprising introducing the shuttle vector of claim 1 into a cell.

13. The method of claim 12 wherein the cell is a Caldicellulosiruptor spp.

14. The method of claim 13 wherein the Caldicellulosiruptor spp. is C. bescii.

15. The method of claim 12 wherein the cell is an E. coli.

16. A genetically modified Caldicellulosiruptor spp. microbe engineered to increase biosynthesis of a bioproduct, wherein the genetically modified Caldicellulosiruptor spp. microbe exhibits an increase in biosynthesis of the bioproduct compared to a wild type control.

17. The genetically modified Caldicellulosiruptor spp. microbe of claim 16 wherein the bioproduct comprises a biofuel.

18. The genetically modified Caldicellulosiruptor spp. microbe of claim 17 wherein the biofuel comprises ethanol.

19. The genetically modified Caldicellulosiruptor spp. microbe of claim 16 wherein the genetically modified Caldicellulosiruptor spp. microbe exhibits an increase in biosynthesis of the bioproduct compared to a wild type control when grown on lignocellulosic biomass.

20. The genetically modified Caldicellulosiruptor spp. microbe of claim 19 wherein the lignocellulosic biomass comprises switchgrass.

21. A method comprising:
growing the genetically modified Caldicellulosiruptor spp. microbe of claim 16 on a feed stock effective for the genetically modified Caldicellulosiruptor spp. microbe to biosynthesize the bioproduct.

22. The method of claim 21 wherein the bioproduct comprises a biofuel.

23. The method of claim 22 wherein the biofuel comprises ethanol.

24. The method of claim 21 wherein the feed stock comprises lignocellulosic biomass.

25. The method of claim 24 wherein the lignocellulosic biomass comprises switchgrass.


SEQUENCE LISTING (56 sequences)

SEQ ID NO: 1 - DNA, 24 bp, artificial
  synthetic oligonucleotide primer
accagcctaacttcgatcattgga

SEQ ID NO: 2 - DNA, 21 bp, artificial
  synthetic oligonucleotide primer
tctgacgctcagtggaacgaa

SEQ ID NO: 3 - DNA, 31 bp, artificial
  synthetic oligonucleotide primer
tctgctagctcaggtcctgctataaagccaa

SEQ ID NO: 4 - DNA, 22 bp, artificial
  synthetic oligonucleotide primer
tcacacgtaccagaaggcagac

SEQ ID NO: 5 - DNA, 19 bp, artificial
  synthetic oligonucleotide primer
cgctaacggattcaccact

SEQ ID NO: 6 - DNA, 20 bp, artificial
  synthetic oligonucleotide primer
tcatctgtgcatatggacag

SEQ ID NO: 7 - DNA, 20 bp, artificial
  synthetic oligonucleotide primer
tccaacgtcatctcgttctc

SEQ ID NO: 8 - DNA, 30 bp, artificial
  synthetic oligonucleotide primer
aagagacgtctcatctgtgcatatggacag

SEQ ID NO: 9 - DNA, 29 bp, artificial
  synthetic oligonucleotide primer
tctgctagctccaacgtcatctcgttctc

SEQ ID NO: 10 - DNA, 33 bp, artificial
  synthetic oligonucleotide primer
agagctagcttcaacaaccagagacacttggga

SEQ ID NO: 11 - DNA, 51 bp, artificial
  synthetic oligonucleotide primer
agcctatcagagaagttcaacaatctagagaccatcctttctatgtagaaa

SEQ ID NO: 12 - DNA, 51 bp, artificial
  synthetic oligonucleotide primer
tttctacatagaaaggatggtctctagattgttgaacttctctgataggct

SEQ ID NO: 13 - DNA, 31 bp, artificial
  synthetic oligonucleotide primer
agagacgtcttaagagattgctgcgttgata

SEQ ID NO: 14 - DNA, 31 bp, artificial
  synthetic oligonucleotide primer
tctggtaccaccgtgagcattctggacaggt

SEQ ID NO: 15 - DNA, 32 bp, artificial
  synthetic oligonucleotide primer
agactcgagattcccatgagcccacgaacagt

SEQ ID NO: 16 - DNA, 33 bp, artificial
  synthetic oligonucleotide primer
agactcgagtcttctgacgctcagtggaacgaa

SEQ ID NO: 17 - DNA, 34 bp, artificial
  synthetic oligonucleotide primer
tctggtaccaccagcctaacttcgatcattggac

SEQ ID NO: 18 - DNA, 56 bp, artificial
  synthetic oligonucleotide primer
agagcatgcgaaaacttgtatttccagggccatcaccatcaccatcactaatttcc

SEQ ID NO: 19 - DNA, 55 bp, artificial
  synthetic oligonucleotide primer
tctggatccaatcctccttttgggattataactgtccatatgcacagatgagacg

SEQ ID NO: 20 - DNA, 39 bp, artificial
  synthetic oligonucleotide primer
agaggatccatgcagataaaggtattgtatgctaacaag

SEQ ID NO: 21 - DNA, 30 bp, artificial
  synthetic oligonucleotide primer
agacagaggtttatgtggttatgggcatgc

SEQ ID NO: 22 - DNA, 37 bp, artificial
  synthetic oligonucleotide primer
tctggtacctctttatcttccattatgagtttgatag

SEQ ID NO: 23 - DNA, 24 bp, artificial
  synthetic oligonucleotide primer
acgttagtcagctgttgttagttc

SEQ ID NO: 24 - DNA, 34 bp, artificial
  synthetic oligonucleotide primer
agaagaacagctgtctgacgctcagtggaacgaa

SEQ ID NO: 25 - DNA, 33 bp, artificial
  synthetic oligonucleotide primer
agaggtaccaccagcctaacttcgatcattgga

SEQ ID NO: 26 - DNA, 35 bp, artificial
  synthetic oligonucleotide primer
tctggtaccagttcctgctttgttaacattccttg

SEQ ID NO: 27 - DNA, 29 bp, artificial
  synthetic oligonucleotide primer
agtgttcttatagctggaattgatacgag

SEQ ID NO: 28 - DNA, 23 bp, artificial
  synthetic oligonucleotide primer
agcgtttgagtatccttttgcag

SEQ ID NO: 29 - DNA, 20 bp, artificial
  synthetic oligonucleotide primer
atccgttgatcttcctgcat

SEQ ID NO: 30 - DNA, 23 bp, artificial
  synthetic oligonucleotide primer
aggatctgaggttcttatggctc

SEQ ID NO: 31 - DNA, 20 bp, artificial
  synthetic oligonucleotide primer
tgggaaagccgtccataatc

SEQ ID NO: 32 - DNA, 20 bp, artificial
  synthetic oligonucleotide primer
tctcccgctcttctctcttt

SEQ ID NO: 33 - DNA, 22 bp, artificial
  synthetic oligonucleotide primer
gtgcgtctacaggaccttattt

SEQ ID NO: 34 - DNA, 21 bp, artificial
  synthetic oligonucleotide primer
ggcaagattctacaggcaaga

SEQ ID NO: 35 - DNA, 21 bp, artificial
  synthetic oligonucleotide primer
tgagcgccaatcaggtataag

SEQ ID NO: 36 - DNA, 21 bp, artificial
  synthetic oligonucleotide primer
ggaagggagatagcggataga

SEQ ID NO: 37 - DNA, 22 bp, artificial
  synthetic oligonucleotide primer
gcatctggtggctatggatatt

SEQ ID NO: 38 - DNA, 20 bp, artificial
  synthetic oligonucleotide primer
acctttgctccacaccttac

SEQ ID NO: 39 - DNA, 24 bp, artificial
  synthetic oligonucleotide primer
tccaatgatcgaagttaggctggt

SEQ ID NO: 40 - DNA, 30 bp, artificial
  synthetic oligonucleotide primer
tctgaattctctgacgctcagtggaacgaa

SEQ ID NO: 41 - DNA, 32 bp, artificial
  synthetic oligonucleotide primer
agaggtacctgtgagggcatgtcaatttacga

SEQ ID NO: 42 - DNA, 34 bp, artificial
  synthetic oligonucleotide primer
agagaattctcttttcgatggaatcttcttcgga

SEQ ID NO: 43 - DNA, 38 bp, artificial
  synthetic oligonucleotide primer
agagagcgatcgtctattgtaactttcacttcagtgca

SEQ ID NO: 44 - DNA, 38 bp, artificial
  synthetic oligonucleotide primer
agaagaaggcggccgctggaagaacttgaaagcaggct

SEQ ID NO: 45 - DNA, 40 bp, artificial
  synthetic oligonucleotide primer
agagagcgatcgacagtttgattacagtttagtcagagct

SEQ ID NO: 46 - DNA, 43 bp, artificial
  synthetic oligonucleotide primer
agaagaaggcggccgcttggttccttaaatctaagaggtatga

SEQ ID NO: 47 - DNA, 21 bp, artificial
  synthetic oligonucleotide primer
tgctggcagagaagagcgaaa

SEQ ID NO: 48 - DNA, 23 bp, artificial
  synthetic oligonucleotide primer
tcttcatcccaatcttcaacttc

SEQ ID NO: 49 - DNA, 32 bp, artificial
  synthetic oligonucleotide primer
actggatccctcaccaaacctccttgtatgat

SEQ ID NO: 50 - DNA, 59 bp, artificial
  synthetic oligonucleotide primer
agagcatgccatcaccatcaccatcactaataataaagctgaaataaaagagggtgaga

SEQ ID NO: 51 - DNA, 38 bp, artificial
  synthetic oligonucleotide primer
actggatccatgacgaaaatagcgaataaatacgaagt

SEQ ID NO: 52 - DNA, 40 bp, artificial
  synthetic oligonucleotide primer
agagcatgctttcttcgcacctccgtaataagcgttcaga

SEQ ID NO: 53 - DNA, 21 bp, artificial
  synthetic oligonucleotide primer
tggtaatgagagaagcagatg

SEQ ID NO: 54 - DNA, 22 bp, artificial
  synthetic oligonucleotide primer
tgataaaaagcacccagtttgt

SEQ ID NO: 55 - DNA, 25 bp, artificial
  synthetic oligonucleotide primer
tggttgaccaggagaattttacaca

SEQ ID NO: 56 - DNA, 23 bp, artificial
  synthetic oligonucleotide primer
agcaacaatcctgcatttgtaag